(19) United States
(12) Patent Application Publication
(10) Pub. No.: US 2023/0076636 A1
(43) Pub. Date: Mar. 9, 2023

(54) AUTOMATED VERIFICATION OF INTEGRATED CIRCUITS

(71) Applicant: Celera, Inc., San Jose, CA (US)

(72) Inventors: Calum MacRae, Charlotte, NC (US); Richard Philpott, Discovery Bay, CA (US)

(21) Appl. No.: 17/894,038

(22) Filed: Aug. 23, 2022

Related U.S. Application Data
(60) Provisional application No. 63/236,589, filed on Aug. 24, 2021.

(51) Int. Cl. G01R 31/319

(52) U.S. Cl. CPC G01R 31/31901; G01R 31/3191

(57) ABSTRACT

Embodiments of the present disclosure pertain to techniques for generating and/or verification of integrated circuits. In one embodiment, parameters of a circuit to be generated are used to automatically generate customized test programs. In another embodiment, an integrated circuit comprises circuits to facilitate testing and controlling test coverage. In yet another embodiment, data obtained from physical circuits is used to generated or modify customized predefined behavioral models of functional circuit components having particular parameters.

### CROSS REFERENCE TO RELATED APPLICATIONS

This application claims priority to U.S. Provisional Patent Application No. 63/236,589, filed on Aug. 24, 2021, entitled “Automated Verification of Integrated Circuits,” the disclosure of which is incorporated herein by reference.

### BACKGROUND

The present disclosure pertains to electronic circuits, and in particular, to techniques for improved test and verification of electronic circuits.

Electronic circuits have become ubiquitous in modern society. From smartphones, personal computers, televisions, and a host of other consumer electronic devices to industrial equipment, scientific instruments, communications systems, and almost every aspect of the Internet, electronic circuits, typically in the form of integrated circuits (or “chips”), have had a dramatic impact on society.

However, developing electronic circuits can be time consuming and costly. Typically, teams of highly trained electrical engineers and a host of the electronic circuit professionals spend long periods of time designing circuit schematics and test and verification systems to build a chip with the desired functionality. Circuit schematics may include each transistor in the circuit together with connections between the transistors. The schematics can be used to simulate how the electronic circuit will operate to ensure proper functionality. Next, the transistors are laid out as geometric shapes. Typically, test systems are developed to test the fabricated chip.

Designing circuit schematics and test/verification systems for an entire electronic circuit can be a tedious and time-consuming process. In particular, for analog circuits, transistor level schematics are typically prepared by very experienced and highly trained analog circuit design professionals. Designing schematics from scratch can take several months depending on the complexity of the circuitry. Additionally, analog circuit test development engineers create a test system for verifying that the circuits behave as desired by the designers. Developing a test system and testing the chips is time consuming and expensive.

Discovering techniques for improving design and test of integrated circuits is advantageous. Additionally, generating schematics and test/verification systems automatically may reduce chip development cycle times, streamline the development process, reduce costs, and provide a wide range of other potential advantages.

### BRIEF DESCRIPTION OF THE DRAWINGS

FIG. 1A illustrates automated verification according to one example embodiment.

FIG. 1B illustrates an example of test routines applied to transistor level schematics of parameterized functional circuit components.

FIG. 2A illustrates automated verification according to another example embodiment.

FIG. 2B is an example of customized tests according to an embodiment.

FIG. 2C is an example of customized tests according to another embodiment.

FIG. 3A illustrates verification of schematics according to another embodiment.

FIG. 3B is an example of verification according to an embodiment.

FIG. 3C is an example of verification according to another embodiment.

FIG. 3D illustrates verification according to another embodiment.

FIG. 4A illustrates a functional circuit component including a test mode according to an embodiment.

FIG. 4B illustrates an example circuit generator and verification system according to another embodiment.

FIG. 4C illustrates generating a circuit according to another embodiment.

FIG. 4D illustrates an example circuit generated according to another embodiment.

FIG. 4E illustrates an example circuit generated when DFT is turned on according to another embodiment.

FIG. 5A illustrates an example of circuitry generated in non-DFT mode according to an embodiment.

FIG. 5B illustrates an example of circuitry generated in DFT mode according to an embodiment.

FIG. 6 illustrates transistor level schematic verification according to an embodiment.

FIG. 7 illustrates integrated circuit testing according to an embodiment.

FIG. 8 illustrates a verification system for parameterized functional circuit components and corresponding circuit modules according to another embodiment.

FIG. 9A illustrates accessing internal nodes of a circuit according to an embodiment.

FIG. 9B illustrates accessing internal nodes of a circuit according to another embodiment.

FIG. 10 illustrates example test circuitry on an analog/mixed signal integrated circuit according to an embodiment.

FIG. 11 illustrates an example of test routines applied to various embodiments of functional circuit components.

FIG. 12A illustrates testing and data collection according to another embodiment.

FIG. 12B illustrates generating a circuit according to an embodiment.

FIG. 12C illustrates generating a circuit according to another embodiment.

FIG. 13 illustrates a computer implemented method according to an embodiment.

FIG. 14 illustrates example computer system hardware according to certain embodiments.

FIG. 15 illustrates various computer system configurations that may be used in certain embodiments.

### DETAILED DESCRIPTION

Described herein are techniques for improved testing and verification of circuits. Some or all of the techniques described herein may be used to make an integrated circuit, for example. In the following description, for purposes of explanation, numerous examples and specific details are set forth in order to provide a thorough understanding of the present disclosure. It will be evident, however, to one skilled in the art that the present disclosure as expressed in the claims may include some or all of the features in these examples, alone or in combination with other features described below, and may further include modifications and equivalents of the features and concepts described herein.

Embodiments of the present disclosure pertain to techniques for generating and/or verification of integrated circuits. In one embodiment, parameters of a circuit to be generated are used to automatically generate customized test programs. In another embodiment, an integrated circuit comprises circuits to facilitate testing and controlling test coverage. In yet another embodiment, data obtained from physical circuits is used to generated or modify customized predefined behavioral models of functional circuit components having particular parameters.

Features and advantages of various embodiments of the present disclosure may include automated verification of circuits. In some example embodiments, transistor level schematics are configured based on parameter values and test routines are automatically generated based on the parameter values to verify the transistor level schematics. In some embodiments, test routines are converted to test cells to verify transistor level schematics. In other example embodiments, the system includes a test mode, where additional circuitry may be included in the transistor level schematics for testing the circuitry. Test routines may include tests that interface with the added test circuitry in the schematics, for example to test one or more circuit modules, for example. In some embodiments, the test routines may further be converted into programs executable on automated test equipment to test physical semiconductor circuits, for example.

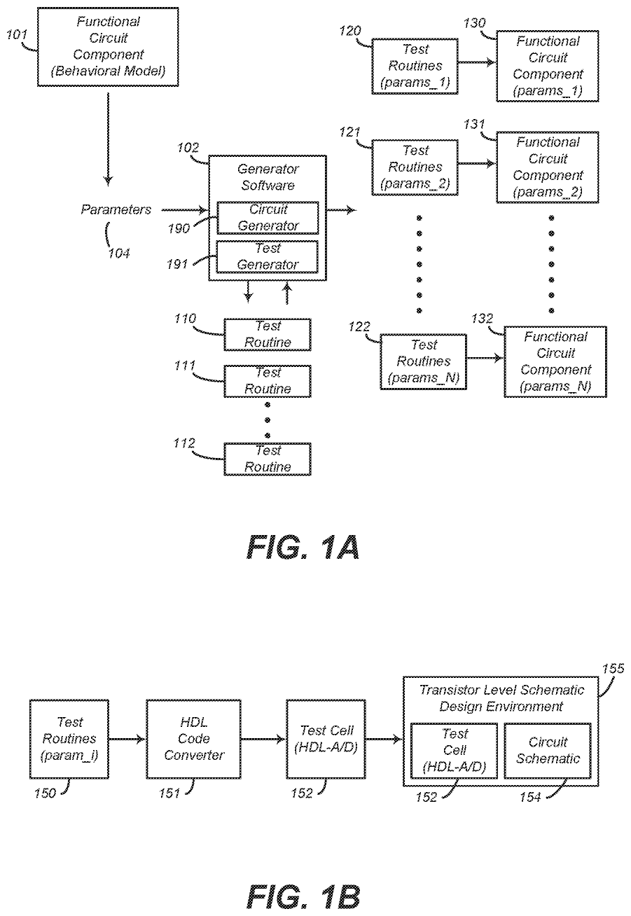

FIG. 1A illustrates automated verification according to one example embodiment. Generator software system (“Generator”) 102 may receive information specifying a plurality of circuit specification parameters 104 corresponding to at least one analog functional circuit component 101 to be created (e.g., into a transistor level schematic or a layout, or both). In this example, generator software 102 includes a circuit generator component 190 for producing transistor level circuits and a test generator component 191 for producing test routines (sometimes referred to as “test macros”). It is to be understood that, in various embodiments, circuit generator 190 and test generator 191 may be part of the same or different software systems. Generating circuits is described in more detail in U.S. patent application Ser. No. 16/882,217, filed May 22, 2020, entitled “AUTOMATED CIRCUIT GENERATION,” the contents of which are hereby incorporated herein by reference in its entirety for all purposes. Generator 102 may process the parameters and generate test routines. For example, generator 102 may select a subset of test routines from a plurality of test routines 110-112 for analog functional circuit component 101. Selection of a subset test routines may be based on (among other things) the parameters (e.g., the same parameters used to select sub-circuits for the functional circuit component), for example. Accordingly, functional circuit components 101 with different parameterized configurations may be automatically tested using different subsets of test routines which are selected based on the same parameters.

For example, the parameters 104 for functional circuit component 101 may have different values corresponding to different configurations of the transistor level schematic for functional circuit component 101 to be generated. A first set of values for parameters 104 (e.g., params_1) may result in generator 102 selecting a first subset of test routines 120 from the plurality of test routines 110-112. The selected test routines may be used to test one transistor level embodiment of functional circuit component 101 configured using the first set of values for parameters 104, such as functional circuit component (params_1) 130, for example. Similarly, a second set of values for parameters 104 (e.g., params_2) may result in generator 102 selecting a second subset of test routines 121 from the plurality of test routines 110-112. Selected subsets of test routines may include some, all, or none of the test routines in other subsets of test routines (e.g., selected subset of test routines 121 may include some, all, or none of the test routines in the subset of test routines 120). The selected test routines 121 may be used to test another transistor level embodiment of functional circuit component 101 configured using a second set of values for parameters 104, such as functional circuit component (params_2) 131, for example. Likewise, other parameter values for parameters 104 (e.g., params_N) may result in other subsets of test routines (e.g., test routines 122) used to test different configurations of transistor level functional circuit components (e.g., component 132 configured by params_N).

As mentioned above and as illustrated further below, features and advantages of some embodiments may use test routines to automatically generate a verification system for a transistor level schematic. FIG. 1B illustrates an example of how test routines may be applied to transistor level schematics of parameterized functional circuit components. In this example, test routines 150 are received by a code converter (or translator) 151. The test routines 150 may have been selected based on a type of functional circuit component and particular parameter values (“param_i”), for example.

In one embodiment, code converter 151 may convert test routines 150 into test cells 152 (e.g., transistor level test scripts), which may be code in a hardware description language for describing analog and/or digital circuits (“HDL-A/D”) such as VHDL or Verilog versions including analog circuits (e.g., Verilog AMS or Verilog A), and which may be executable by an electronic design automation (EDA) system, for example. In one embodiment, test cells 152 may be imported into an electronic design automation (EDA) system 155, such as Cadence® or Mentor®, for example, and used to verify the functionality of an analog transistor level circuit schematic 154 corresponding to one or more parameterized functional circuit components.

Accordingly, test cells 152 and circuit schematic 154 may, in some example embodiments, be selected based on at least some of the same particular parameter values (e.g., param_i) so that as the schematic changes based on different parameter values (e.g., as pins change or as functionality of the circuit changes—gain, maximum current, clamps, input/output voltage or current levels, etc. . . . ) the test cells also automatically change to match the schematic changes and verify the functionality of different transistor level schematics being generated. In various embodiments, different parameters and/or parameter values may select different test routines (e.g., including different parameters for selected test routines), which will be converted into different test cells 152 for testing circuits generated using the same set of parameters, for example. In some example applications, an integrated circuit with multiple parameterized functional circuit components may have test routines corresponding to each parameterized functional circuit component selected and converted into a test cell for testing the transistor level schematic of the integrated circuit in an EDA environment, for example. Verification in the EDA environment may be advantageous in ensuring the integrated circuit works as intended and may reduce testing costs of the physical circuitry, for example.

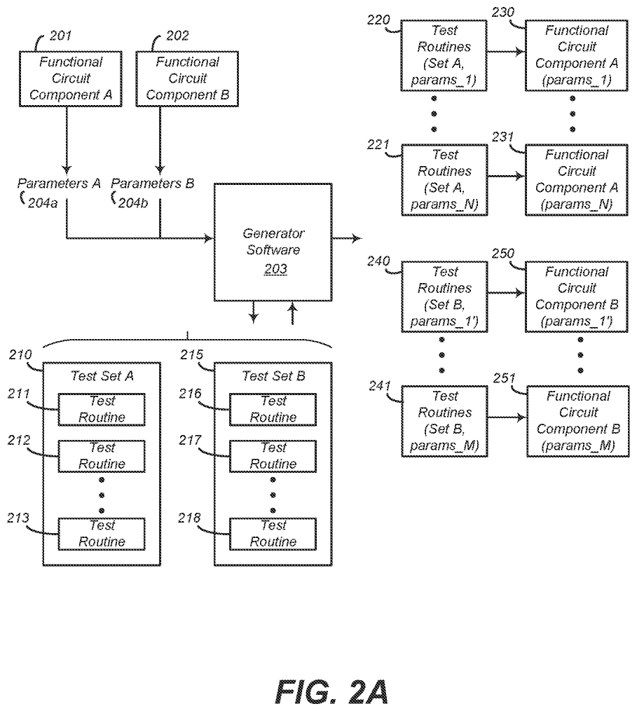

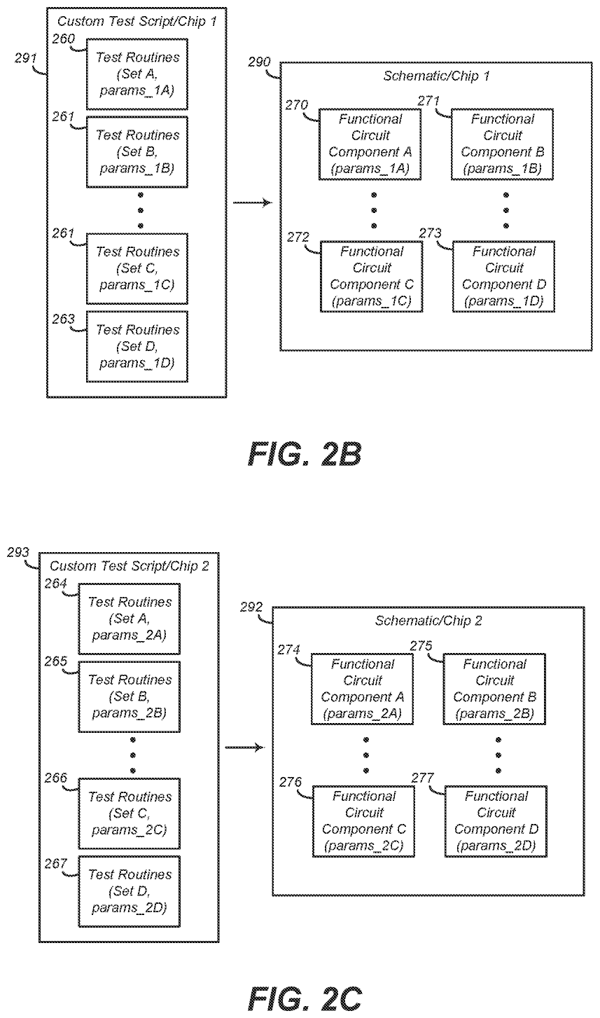

FIG. 2A illustrates automated verification according to another example embodiment. In some embodiments, different functional circuit component types may have different parameters and corresponding test routines. In this example, a first functional circuit component (Component A, e.g., a voltage to current converter) 201 may have a first set of parameters (Parameters A) 204a and a second functional circuit component (Component B, e.g., a voltage buffer) 202 may have a second set of parameters (Parameters B) 204b. Examples of different types of functional circuit components may include amplifiers, buffers, comparators, oscillators, power switches, resistor dividers, feedback dividers, voltage to current converters, current sources, current generators, current mirrors, and a wide range of other analog/mixed signal circuit components, for example. Different functional circuit component types may have different groups of test routines to select from. For example, functional circuit component A may have corresponding parameters 204a for selecting subsets of test routines from test set A 210, which may include test routines 211-213. Similarly, functional circuit component B may have corresponding parameters 204b for selecting subsets of test routines from test set B 215, which may include test routines 216-218, for example. Test set A 210 may comprise a plurality of tests for testing functional circuit component A 201 across a range of values for parameters 204a. Similarly, test set B 215 may comprise a plurality of tests for testing functional circuit component B 202 across a range of values for parameters 204b.

Accordingly, in some example embodiments, different parameter values for a functional circuit component may result in selection of different test routines that may be used to test the different resulting circuitry. For example, a first set of parameters (Parameters A) 204a may be used to produce a transistor level schematic for functional circuit component A 201. Accordingly, a first subset of test routines 220 are selected from a first plurality of test routines in test set A 210 based on one or more values of parameters 204a (e.g., Set A, params_1), for example. Test routines 220 may be used to test an embodiment of functional circuit component A 230 (e.g., a transistor level schematic or physical IC) having parameters 204a with values params_1, for example. As mentioned above and as described in more detail below, selected test routines may be used to test either or both of a transistor level schematic or a physical semiconductor device, for example. A second subset of test routines 221 are selected from test set A 210 when generator 203 receives different values for parameters A 204a (e.g., denoted here as Set A, params_N) for functional circuit component A 201. Thus, test routines 221 may be used to test another embodiment of functional circuit component A 231 having parameters “param_N,” for example.

As another example, a second set of parameters (Parameters B) 204b may be used to configure another functional circuit component B 202. Accordingly, a first subset of test routines 240 are selected from a second plurality of test routines (e.g., test set B 215) based on one or more values of parameters 204b denoted here as “Set B, params_1′,” for example. Test routines 240 may be used to test an embodiment of functional circuit component B 250 (e.g., a transistor level schematic or physical IC) having parameters 204b with values of “params_1′,” for example. A second subset of test routines 241 are selected from test set B 215 when generator 203 receives different values for parameters B 204b (e.g., denoted here as Set B, params_M) for component B 202. Test routines 241 may be used to test another embodiment of functional circuit component B 251 having parameters “params_M,” for example.

FIGS. 2B-C illustrate one advantage of some embodiments. In this example, a first set of parameter values for a plurality of functional circuit components are used to generate an analog circuit schematic (and chip) 290 including functional circuit components 270-272, for example, each being generated based on particular parameter values. Advantageously, the parameter values are also used to generate a custom test script 291 comprising customized test routines 260-263 selected based on the parameter values (and possibly other criteria). Accordingly, the parameters may be used to generate both a custom schematic (and chip) and a custom test script, which may be converted (as described above) into an HDL script for verifying the transistor level schematic and/or into a test program executable on a physical test system for testing the physical IC (as described in more detail below). Likewise, a second set of parameters for the functional circuit components may be used to generate another schematic (and chip) 292 and a custom test script 293. It is to be understood that a wide variety of schematics (and corresponding chips) may be generated with different functional circuit components and different parameters based on the desired chip to be produced and the present techniques automatically produce customized test scripts. The test scripts may be converted to execute in an EDA environment to verify the transistor level schematics, for example, and/or be converted into test programs for testing the physical chips, which may significantly reduce the amount of development time needed to produce an analog/mixed signal integrated circuit, for example.

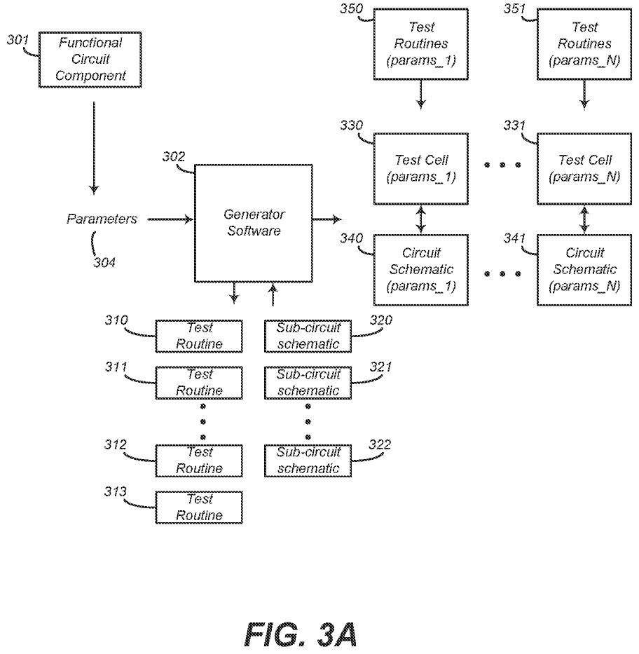

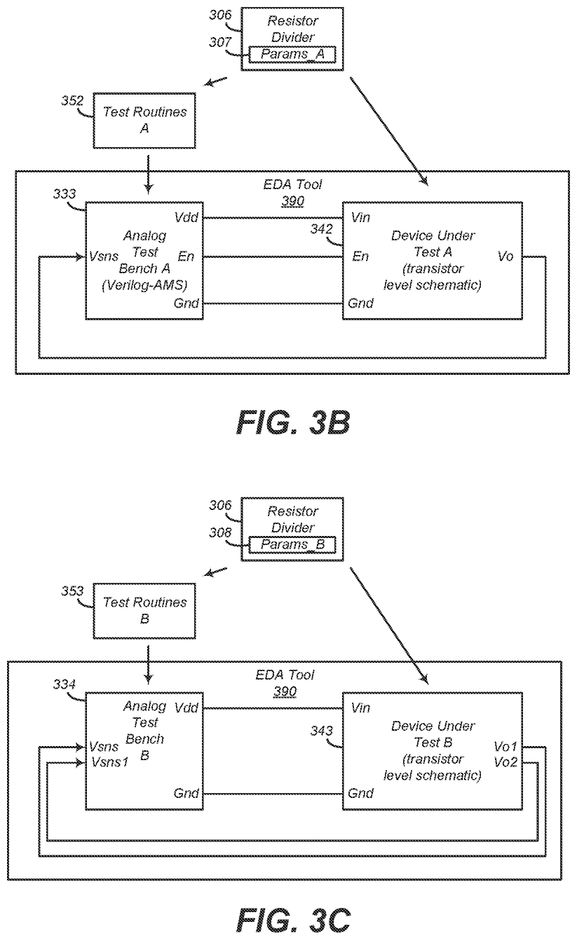

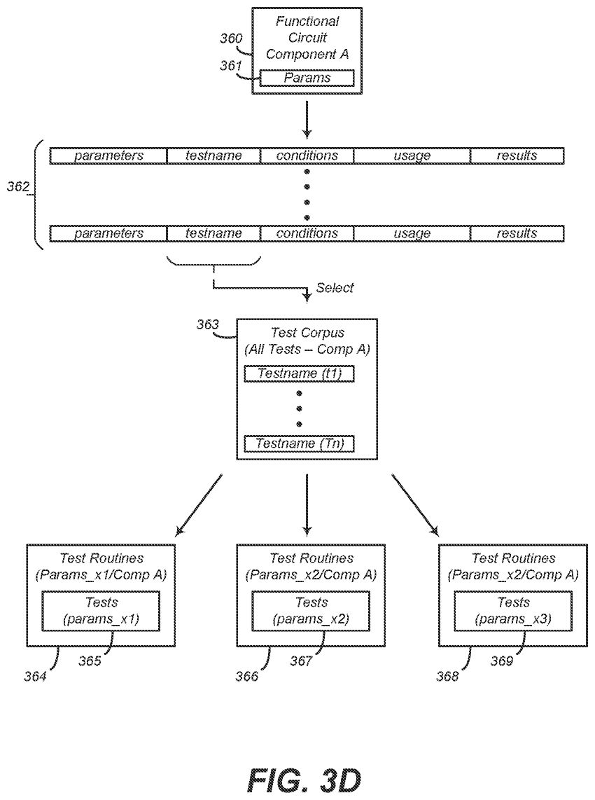

FIG. 3A illustrates verification of schematics according to another embodiment. In this example, parameters 304 for functional circuit component 301 may be used to select a subset of analog sub-circuit schematics 320-322 (e.g., as described above) and a subset of test routines 310-313. For instance, functional circuit component 301 may have a corresponding plurality of analog sub-circuit schematics 320-322, which may be combined in different combinations based on parameters 304 to form different circuit schematics for functional circuit component 301 having different parameterized characteristics (e.g., comparator hysteresis external or internal, deglitch on or off, n-type or p-type, oscillator frequency ranges, bias current values, gain values, clamp circuit or no clamp circuit, etc. . . . ). Accordingly, different combinations of analog sub-circuit schematics for a particular type of functional circuit component with different circuit specification parameter values may have different subsets of test routines. For example, when generator software 302 receives a first set of parameter values denoted here as “params_1,” a first subset of sub-circuit schematics 320-322 may be selected and combined to form a first circuit schematic 340. Additionally, generator software 302 may select a first subset of test routines 350 from test routines 310-312 using one or more of the same first set of parameter values “params_1,” for example. Features and advantages of the present disclosure include using the selected subset of test routines 350 to generate one or more test cells 330, which may be used to test circuit schematic 340. Circuit schematic 340 may have features or characteristics specified by the first set of parameters, “params_1.” Since, in this illustrative example, the test routines may be selected based on the same parameter values used to select the sub-circuit schematics combined to form circuit schematic 340, the selected test routines (e.g., including parameters of the test routines, such as applied voltages and/or measurements on particular pins) may be configured to match the particular combination of analog sub-circuit schematics used in the circuit schematic 340. Accordingly, a variety of different circuit schematic features, specified by the different parameter values, may be tested by test routines customized for such features. As yet another example, generator software 302 may receive a second set of parameter values denoted here as “params_N.” Based on parameter values “params_N,” generator 302 may select sub-circuit schematics to generate circuit schematic 341, and generator 302 may also select a subset of test routines 351 from test routines 310-313, which are converted to test cells 331 customized to test circuit 341, for example.

As illustrated in FIG. 3A, the test cells 330 and 331 may be coupled to one or more of the sub-circuit schematics to test the circuit schematics 340 and 341, respectively, in a transistor level schematic EDA tool (e.g., using a transistor level simulator), for example. For instance, test cell 330 may specify voltage or current sources, voltage or current measurement components, waveform generators, waveform measurement components, and other analog and digital signal generators used to test circuit schematic 340, respectively, for example. Test cell 330 may include analog terminals or digital terminals, or both, that are coupled to analog and/or digital terminals of the circuit schematic 340 being tested. Output terminals of test cell 330, which may be used to generate voltages, currents, or other analog or digital signals (e.g., in a simulation mode), may be coupled to input terminals of circuit schematic 340, and input terminals of test cell 330 may be coupled to output terminals of circuit schematic 340 to receive voltages, currents, or other analog or digital signals. Signals received on input terminals of test cell 330 may be measured (e.g., in a simulation mode) to verify that circuit schematic 340 is working as intended. While the circuit schematics 340 and 341 may be implemented using a plurality of primitive components, such as transistors, resistors, and/or capacitors, for example, test cell 330 and 331 may be implemented in an HDL with analog, such as Verilog-AMS or Verilog-A, for example, and as mentioned above in connection with FIG. 1B. Test routines 350 and 351 may be written in a variety of programming languages and converted to Verilog-AMS or Verilog-A. Converted test routines may result in test cells, which when executed in an EDA environment, for example, result in a sequence of voltages and/or currents to be applied to a circuit schematic. The simulation results may be advantageously used to verify that the circuit schematic works as desired.

FIGS. 3B-C illustrate an example of verification according to an embodiment. In FIG. 3B, a resistor divider functional circuit component 306 is configured with a first set of parameter values 307 (“params_A”). Parameter values 307 may correspond to a particular resistor divider resistance and a number of taps, for example. Parameter values 307 and their particular values may be used to select test routines A 352 and generate a transistor level schematic of a device under test A 342 (here, a particular resistor divider). Test routines A 352 may be converted to a test cell (analog test bench 333), which may be an analog/mixed signal HDL, such as Verilog-A, for example. In this case, device under test 342 is a resistor divider having a voltage input pin, Vin, ground pin, Gnd, an enable input pin, EN, and one output tap pin, Vo. Test bench 333 includes an analog voltage pin, Vdd, for forcing voltage (and possibly measuring current), for example, a ground pin, Gnd, an enable pin, EN, and a voltage sense pin, Vsns. The selected test routines 352 may be converted to HDL code to configure test cell 333 to generate a voltage on Vdd (and Vin) and measure current as part of a test to measure the resistance of the resistor divider, for example. Another test in test routines 352 may force a voltage on Vdd(Vin) and measure the voltage on Vsns(Vo), for example. The following is an example automatically generated test routine for testing a disable feature, resistance, and voltage division of a resistor divider, which may be converted to a test cell for testing a schematic in an EDA environment or a test program for testing physical resistor divider circuits.

```
// Example Test Routine - Resistor Testing
srccfg VDD step 0 1u 100
srccfg GND step 0 1u 100
srccfg EN dig 1 0
probe Vsns
begin
  src GND 0.0 1u
  call test_shutdown 5 0 //input conditions VDD=5v, EN=0
  call test_resistance 5 //input conditions VDD=5v
  call test_divider_voltage 5 //input conditions VDD=5v
end
#macro test_shutdown //input parameters: VDD=$1 and EN=$2
  src VDD $1 1u
  src EN $2
  check_current VDD //measure current in Vdd pin
#endmacro
#macro test_resistance //input parameters: VDD=$1
  src VDD $1 1u
  src EN 1
  check_current VDD
#endmacro
#macro test_divider_voltage //input parameters: VDD=$1
  src VDD $1 1u
  src EN 1
  check Vsns
#endmacro
```

In this example, a test routine includes source definitions (e.g., srccfg) for analog pins VDD and GND and a digital enable input EN. The routine further includes probe pin definition (e.g., probe) Vsns for receiving an analog voltage. It is to be understood and a variety of other source (output) and probe (input) definitions could be used in various embodiments. The test routine further includes subroutines (here, “macros”) for each test. The main body of the test routine is between the “begin” and “end” statements, and macro calls for the desired tests are selected with appropriate conditions as input parameters for the various tests. In this example, sources are set using the “src” command and probes are measured using the “check” command. Subroutines/macros may be pre-defined and stored (e.g., in a database) and selectively retrieved and loaded into a test script based on parameters (and other criteria described below), for example. In various embodiments, source definitions, probe definitions, macros, and macro input parameters (conditions) may be predefined and selectively loaded into a test routine automatically based on one or more of, for example, functional circuit component type, functional circuit component parameters, conditions (e.g., default, user defined, calculated, or parameter based as described in more detail below), and/or usage (described further below). Accordingly, a user may select a circuit design comprising a variety of functional circuit components, and customized test routines for the selected circuit design may be generated automatically based on the above factors as well as user input, if any, for EDA verification or testing of physical circuits, for example.

In FIG. 3C, the resistor divider functional circuit component 306 is configured with a second set of parameter values 308 (“params_B”). Parameter values 308 may correspond to another resistor divider resistance and/or a different number of taps (e.g., two (2) taps), for example. Parameter values 308 may be used to select test routines B 353 and generate a transistor level schematic of a device under test B 343 (e.g., a resistor divider with two (2) taps). Test routines B 353 may be converted to an analog test bench 334 customized for the new parameterized circuit schematic, for example. In this case, device under test 343 is a resistor divider having a voltage input pin, Vin, ground pin, Gnd, and two output tap pins, Vo1 and Vo2. Test cell 334 includes an analog voltage pin, Vdd, for forcing voltage (and possibly measuring current), for example, a ground pin, Gnd, and two voltage sense pins, Vsns and Vsns1. The selected test routines 353 may be converted to HDL code to configure test cell 334 to measure the resistance of the resistor divider and the voltages on Vsns/Vo1 and Vsns/Vo2, for example. Automated test generation is illustrated here using a simple case of resistor dividers. However, it is to be understood that the testing techniques described herein may be used on a wide range of other more complex parameterized circuits.

FIG. 3D illustrates verification according to another embodiment. In some embodiments, parameters and test conditions may be used to select tests. For example, functional circuit component A 360 may be configured with parameters 361. Parameters 361 may be associated with one or more of the following: particular tests (e.g., testnames), test conditions, usage, and/or results. As mentioned above, parameters may be used to select test routines from a set of test routines for a component. In this example, particular parameter values may be associated one or more testnames, which may be used to select test routines from a set of test routines. Additionally, particular tests may be performed under certain test conditions (e.g., particular voltages, currents, frequencies, times, etc. . . . ), which may be user defined, predefined (e.g., defaults), calculated, and/or based on one or more parameter values. Accordingly, in some embodiments, a particular testname may further be associated with test conditions for the test routine. For example, an input threshold test (e.g., associated with a comparator having parameters specifying a one threshold) may be associated with conditions to specify a first input voltage to be used to test the threshold. Another instance of the input threshold test (e.g., associated with a comparator having parameters specifying a different threshold) may be associated with conditions to specify a second input voltage to be used to test the different threshold, for example. As another example, a set of parameter values for an amplifier functional circuit component may be used to select a gain test performed using one input voltage condition (e.g., if parameter “AmpGain”=1), while another set of parameter values for the amplifier component may be used to select a gain test performed using another input voltage condition (e.g., if parameter “AmpGain”=10). Accordingly, gain tests may be performed across a range of parameterized gains for different amplifiers without causing the device output to reach a rail voltage, for example.

In some embodiments, particular test routines may be associated with a “usage.” A usage for a test may specify different instances where particular test routines are used. For example, some tests may be selected and included in a test routine for “characterization” of a circuit (e.g., running tests across a wide range of conditions to gain an understanding of performance or potential error or failure modes). Other tests may be performed to determine “typical” performance results, where the results of such tests are in a data sheet, for example. Yet other tests may be used in a production environment (e.g., when testing a physical semiconductor device) comprising a variety tests that may or may not be used in a data sheet or in characterization. Further, yet other tests may be used in an automated lab bench test environment (e.g., to bench test particular features of the device). Accordingly, the usage indicator may also be used to automatically select test routines and conditions for one of a plurality of different test environments (e.g., EDA schematic testing, bench testing, characterization, production, or typical/data sheet). For instance, specific tests may be selected (e.g., specific testnames) with specific conditions for each test based usage fields to automatically produce a list of tests to be performed during characterization, for example, while another set of tests may be selected with the same or different specific conditions for each test based usage fields to automatically produce a list of tests to be performed during production.

Additionally, in some embodiments, test routines may be associated with one or more particular test results (e.g., expected test results). In some cases, the results may indicate a range (e.g., maximum and minimum limit values) that the test result must be within for the test to pass. In other cases, the test results may specify that the test is pass/fail, wherein the test routine may apply certain inputs and analyze one or more outputs for the occurrence of a condition or event (e.g., is the output voltage high or low; if high, then pass, else fail).

In one example implementation, as illustrated in FIG. 3D at 362, combinations of parameter values may be associated with one or more different records comprising a plurality of fields (e.g., in a database, spreadsheet, file, or other data structure). For example, each record may correspond to a unique set of parameter values for a functional circuit component, and may include fields for one or more parameters as well as associated fields for testname, conditions, usage, and/or results, for example.

In this example, the testname field may be used as a key for selecting test routines from a plurality of test routines. Here, a test corpus 363 may store test routine code (e.g., calls and implementing subroutines—e.g., macros) for all the test routines (e.g., which may include each test setup and measurement) used to test functional circuit component 360 across all the values of parameters 361 (e.g., and optionally all usages). The testnames (e.g., t1 . . . tN, for N tests) may be the same as the testnames stored in the records 362, for example. Parameter values may be used to select a subset of testnames (optionally also based on usages), and the resulting testnames may be used to select a subset of test routines from the corpus of all test routines 363 for functional circuit component A, for example. Conditions associated with particular parameter values and/or tests may be applied during execution, for example. While testnames are used as the key to link parameters and test routines in this example, it is to be understood that other keys for linking parameters, test routines, and/or the above mentioned fields may be used.

Therefore, in this example, one set of parameters (e.g., params_x1) for functional circuit component A 360 (aka “Comp A”) may produce a first set of test routines 364 comprising tests 365 (e.g., and specific conditions) selected as a function of params_x1. Similarly, a second set of parameters (e.g., params_x2) for functional circuit component A 360 may produce a second set of test routines 366 comprising tests 367 selected as a function of params_x2. Here, a third set of parameters (e.g., params_3) for functional circuit component A 360 may produce a third set of test routines 368 comprising tests 369 selected as a function of params_x3. As mentioned above, test routines 364, 366, and 368 may be used to test transistor level schematics and/or physical semiconductor circuits.

Some example embodiments of the present disclosure may include test routines that may be performed on a physical device. For example, some transistor level circuit modules generated from particular parameterized functional circuit components may be verified using transistor level simulations, as described above. However, when circuit modules for functional circuit components are combined with a plurality of other circuit modules for other parameterized functional circuit components, it may be more challenging to perform the same tests used when testing the individual circuit modules alone (e.g., testing a design with a comparator, amplifiers, current mirrors, resistor divider, etc. . . . may be more difficult than testing a comparator alone). For instance, certain nodes may not be accessible when a plurality of circuit modules are combined on the chip.

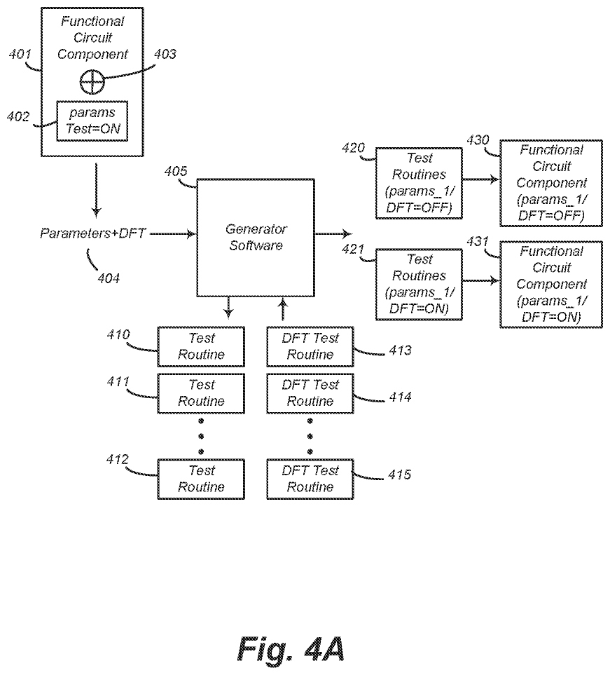

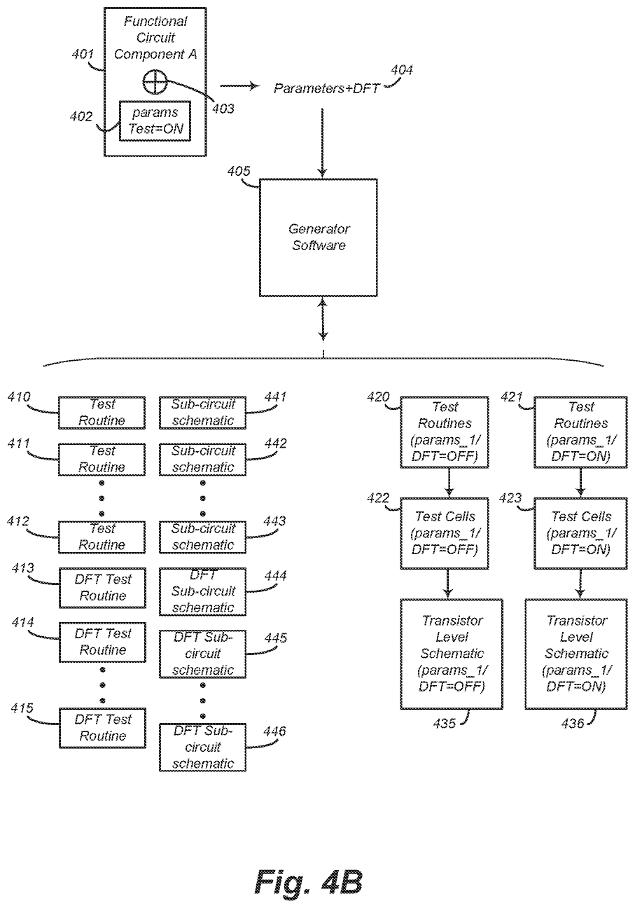

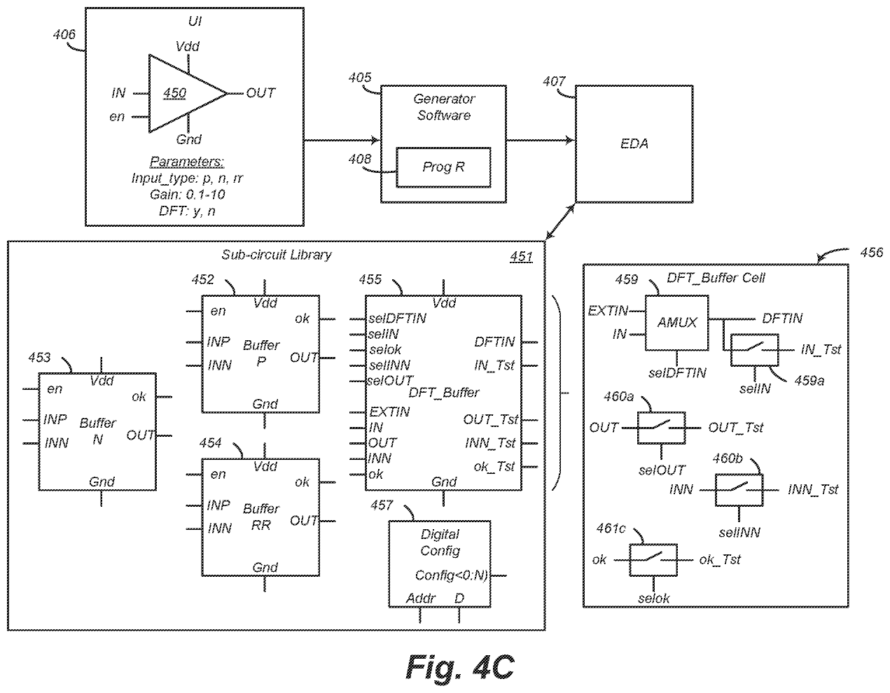

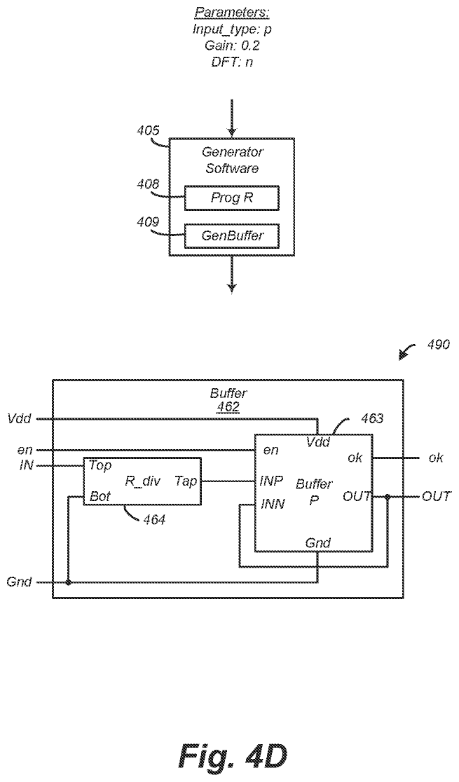

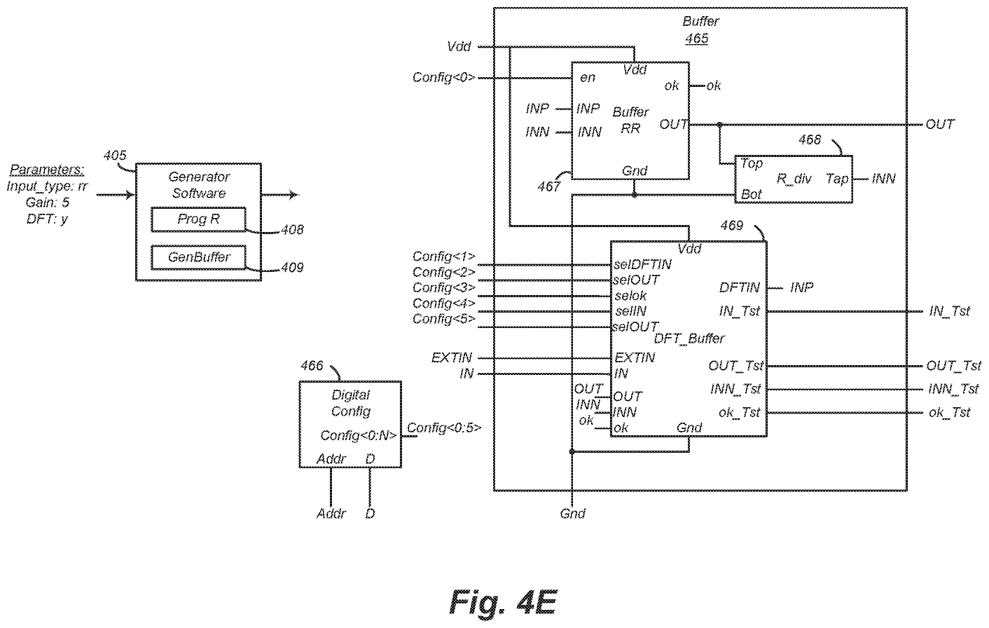

Features and advantages of the present disclosure further include test routines and additional circuitry for testing circuit modules when such modules are combined with other circuit modules in an integrated analog or mixed signal circuit, for example. FIG. 4A illustrates a functional circuit component 401 that includes a test mode. Test mode may be at least one test parameter 402 of the functional circuit component (e.g., in addition to other parameters for the functional circuit component described above). Test parameter 402 may be referred to herein as “design for test” (DFT or “test mode”), and a plurality of functional circuit components may have a DFT parameter activated or deactivated (DFT=Yes or No). When DFT is active, additional circuitry may be added to the functional circuit component to allow direct testing of the resulting circuitry even if the component's circuit inputs and outputs are embedded in a circuit design. As mentioned above, in some example embodiments, functional circuit components may be displayed to a user in a user interface. When a user turns DFT on or off, an indication 403 (here, a circle with cross bars) that the component is in DFT mode is displayed. In some example embodiments, as a user builds a design in a UI using parameterized functional circuit components, the user may turn DFT on and off across their design to set which functional circuit components they want to test directly. Accordingly, the UI may show a test coverage across a plurality of interconnected functional circuit components for the composite analog circuit to the user.

In one embodiment, when DFT is turned on, different tests may be selected for a functional circuit component versus when DFT is turned off. For example, generator software 405 may receive parameters 404, which include a DFT parameter. When DFT is off (DFT=No), then generator 405 may select one or more test routines 410-412 as described above (e.g., for normal, or non-DFT, mode). However, when DFT is on (DFT=Yes), generator 405 may select one or more DFT test routines 413-415 based on the above described techniques and further based on the value of the DFT parameter. Accordingly, for a particular set of parameter values, denoted “params_1”, generator 405 may select test routines 420 to test embodiments of functional circuit component 430 when DFT is off, and generator 405 may select test routines 421 to test embodiments of functional circuit component 431 when DFT is on. Selection of such routines may include either selection of specific DFT and Non-DFT tests, for example. Different test routines for DFT on and off may be advantageous when testing variations of a particular functional circuit component with different parameters directly and individually in a EDA environment. As illustrated in various examples below, the test routines for testing particular functional circuit components with DFT on and DFT off may be different in some cases.

Further advantages of DFT mode include the ability to apply test routines to verify one or many parameterized analog circuit modules embedded in a chip design.

FIG. 4B illustrates an example circuit generator and verification system according to another embodiment. In some embodiments, additional circuitry may be included in a transistor level schematic when the test mode is active (e.g., DFT=Y). In this example, generator software 405 receives parameters 404, which includes a DFT parameter, for a functional circuit component 402 as described above in FIG. 4A, and selects one or more DFT test routines 413-415 based on parameters 404 when DFT is on. In this example, when DFT is on, generator 405 may select a different subset of sub-circuit schematics 441-446. For example, with DFT off, a particular set of parameter values, “params_1”, may result in a transistor level schematic of a circuit 435 being generated from sub-circuit schematics selected from 441-443. However, when DFT is on, a transistor level schematic of a circuit 436 may be generated (e.g., based on values of parameters 404) that includes one or more DFT sub-circuit schematics 444-446. In some instances, the same sub-circuit schematics 441-443 used when DFT is off may be used when DFT is on (although the connections may be different). However, in some cases one or more of the sub-circuit schematics 441-443 used when DFT was off may be substituted for DFT sub-circuit schematics. Therefore, in some embodiments, one or more DFT sub-circuit schematics may be added to a selected set of non-DFT sub-circuit schematics. In other cases, one or more non-DFT sub-circuit schematics may be replaced with a corresponding DFT sub-circuit schematic that perform substantially the same function but further includes additional DFT circuitry, for example. As illustrated in more detail below, DFT circuitry may include one or more analog switches to allow analog voltages, currents, or AC waveforms to be applied to, or sensed from, input nodes, output nodes, or even internal nodes of circuit modules in a circuit comprising a plurality of analog or mixed signal circuit modules, where the circuit modules may each have been generated from corresponding functional circuit components, for example.

With respect to the test routines, when DFT is off, selected test routines 420 may be converted to test cells 422. Test cells 422 may be used to verify transistor level schematic 435 for a circuit module generated from functional circuit component 401 (e.g., directly in an EDA environment). In this example, when DFT is on, test routines 421, selected as described above, may be converted to test cells 423 and used to verify transistor level schematic 436, which may include DFT sub-circuits, for example. Advantageously, with DFT on, transistor level schematic 436 may be tested directly using test cell 423 in an EDA environment or transistor level schematic 436 may be tested when it is embedded in a chip either in an EDA environment or a physical chip, for example.

FIG. 4C illustrates generating a circuit according to another embodiment. In this example, a user may select a buffer functional circuit component 450 in a user interface (UI) 406. Buffer 450 in UI 406 may include parameters for input_type (e.g., n-type (n) transistor input stage, p-type (p), or rail-to-rail (rr)), gain (e.g., 0.1-10), and DFT (yes or no), for example. Buffer 450 may have corresponding transistor level sub-circuit schematics in a sub-circuit library 451. Sub-circuits for buffer 450 may include a p-type input stage buffer circuit 452, an n-type input stage buffer circuit 453, and a rail-to-rail input stage (e.g., both p and n input devices) 454. Additionally, generator 405 may invoke programmable resistor (“Prog R”) module 408 (e.g., as described above) to automatically generate resistors for a resistor divider, for example, to set the gain of the buffer. Further, library 451 may include DFT circuits including a DFT_Buffer sub-circuit 455 and a digital configuration circuit 457. DFT_Buffer 455 may include pins for analog input/output signals (e.g., buffer input voltage (IN), external buffer input voltage (EXTIN), a buffer output voltage (OUT), a voltage at an internal input node of the buffer (INN), DFTIN, IN_Tst, OUT_Tst, INN_Tst), digital pins for digital signals (e.g., a buffer ready signal (ok) and ok_Tst), and configuration pins for configuration signals (e.g., selection signals selDFTIN, selIN, selok, selINN, selOUT).

Circuits inside the DFT_Buffer 455 are illustrated at 456. DFT_Buffer 455 includes an analog multiplexer (AMUX) 459 having inputs coupled to EXTIN and IN and a select input coupled to selDFTIN. An analog multiplexer is a multiplexer configured to select and pass analog signals (e.g., an input is selectively coupled to an output through one or more pass transistors). The output of AMUX 459 is coupled to DFTIN. The output is also coupled through a switch 459a, which has a control input coupled to selIN, to IN_Tst. Accordingly, AMUX 459 may couple either an input of a previous stage (e.g., IN, during normal operation) or an external input (e.g., EXTIN, during a test mode) to the DFTIN pin, which may be coupled to the buffer input when DFT is “on” as described further below. Additionally, the buffer input voltage (e.g., IN or EXTIN) may be sensed (e.g., during testing) by activating selIN and sensing IN_Tst, for example. Advantageously, when a parameterized buffer circuit is embedded in a chip and DFT is “on,” EXTIN and IN_Tst may be accessible externally to the chip to allow testing of the buffer inside a larger design, for example.

In this example, DFT_Buffer 456 further includes a switch 460a having an input coupled to OUT (an output of the buffer stage), a select input coupled to selOUT, and an output OUT_Tst. Accordingly, when selOUT is set the output (OUT) of the buffer may be sensed (during testing) to determine a voltage on the output of the buffer, for example. OUT_Tst may be accessible externally to the chip as mentioned above. DFT_Buffer 456 further includes a switch 460b having an input coupled to INN (a negative input of the buffer stage), a select input coupled to selINN, and an output INN_Tst, which may be sensed (e.g., externally) to determine a voltage on the negative input of the buffer, for example. DFT_Buffer further includes a switch 460c having an input coupled to digital signal “ok” (a buffer “on” signal indicating the buffer is powered up and operational), a select input coupled to selok, and an output ok_Tst, which may be sensed to determine if the buffer is turning on, for example. It is to be understood that the above buffer circuit, pins, connections, and DFT circuitry are merely examples of how DFT may incorporate additional DFT circuitry to allow direct testing of parameterized functional circuit components embedded in an analog/mixed signal chip design. One or more of the same or similar example techniques illustrated here may be implemented for a variety of other functional circuit components to directly input or directly output voltages, currents, analog signals, or digital signals, for example.

FIG. 4D illustrates an example circuit generated according to another embodiment. In this example, a user has selected buffer parameters as follows: input_type=p, gain=0.2, and DFT=n. Generator software 405 includes a generate buffer module 409, which may access a generate resistor module 408. Generator software 405 receives the parameters and may calculate values for a resistor divider (to produce the desired gain) and generates a netlist specifying a plurality of resistors and a p-type buffer 452 with the pins mapped as shown at 490. The netlist may be received in an EDA tool, for example, which generates a buffer symbol 462 with an instance of the p-type buffer 463 and a resistor divider 464 inserted inside with terminals connected together as shown. In this simplified example, generate buffer module 409 may determine, based on a gain of less than one (e.g. gain=0.2) that the resistor divider 464 is to be mapped between input pin IN of the symbol and INP of a p-type input stage buffer circuit 463. Buffer module 462 may be tested directly in an EDA environment and/or embedded in a design where direct testing of the buffer inputs and outputs may not be possible.

FIG. 4E illustrates an example circuit generated when DFT is turned on according to another embodiment. In this example, a user has selected buffer parameters as follows: input_type=RR, gain=5, and DFT=y. Generator software 405 receives the parameters and may calculate values for a resistor divider (to produce the desired gain) and generates a netlist specifying a plurality of resistors, (referring again to FIG. 4C) a RR-type buffer 454, a DFT_Buffer 456, and a digital configuration circuit 457 with the pins mapped by generate buffer module 409. The netlist may be received in an EDA tool, for example, which generates another buffer symbol 465 including instances of the RR-type buffer 467, a resistor divider 468, and DFT_Buffer 469. An instance of digital configuration circuit 466 may further be generated. Digital configuration circuit 466 may be internal to buffer 465 in some embodiments, although it is illustrated here as being external, for example. In this example, generate buffer software module 409 in generator 405 generates a netlist with the pins mapped as shown. Here, generate buffer module 409 may determine, based on a gain greater than one (e.g. gain=5), that the resistor divider 468 is to be mapped between output pin OUT of the buffer symbol 465 (connected here to RR buffer 467 OUT) and negative input INN of the buffer circuit 463 (e.g., R_div/Tap:INN).

From FIG. 4E it can be seen that turning DFT on adds circuitry and terminals to the buffer symbol 465. In this example, an additional external (or auxiliary) input, EXTIN, is included. Further, with DFT enabled Buffer 465 comprises additional analog outputs, including IN_Tst (here, for sensing the positive input, INP, of RR buffer 467, which is connected to DFTIN), OUT_Tst (e.g., for sensing the output of the buffer), INN_Tst (e.g., for sensing the negative input, INN, here, the feedback voltage), an additional digital output (e.g., ok_Tst for sensing the internal “ok” signal on the buffer), and configuration inputs (e.g., config<0:5> for controlling the enable pin, “en,” and selection of the DFT analog input and output signals). Finally, an instance of digital configuration circuit 466 is generated. Digital configuration circuit 466 may include, for example, an address input and data (D) input. Digital configuration circuit 466 may receive data (D) sent to it and produce digital values on the config< > lines based on the data to selectively control DFT_Buffer 469 or selectively enable buffer 465 (or both), for example. Accordingly, during testing, external input signals may be applied to the buffer, and outputs of the buffer may be sensed externally. The same or substantially similar techniques may be used for testing other functional circuit components. By turning DFT “on,” functional circuit components may be tested individually even when embedded in a chip design. Accordingly, analog circuit testing may be conducted from the “inside out,” rather than the traditional “outside in” where the entire chip circuitry is tested as a whole using exposed inputs and outputs. Additionally, as mentioned above, some embodiments may allow users to selectively turn on DFT for certain functional circuit components used in an analog or mixed signal design while turning DFT off for other functional circuit components in the design. As the number of circuit modules with DFT on increases, the test coverage increases but the test time and cost go up. Similarly, as the number of modules with DFT on decreases, the test coverage decreases but the test time and cost go down. The disclosed technique of turning DFT on and off for each functional circuit component allows designers to advantageously determine the cost/benefit tradeoff of additional circuitry and test time versus test coverage across the design as a whole, for example, and create customized test coverages optimized for each chip design.

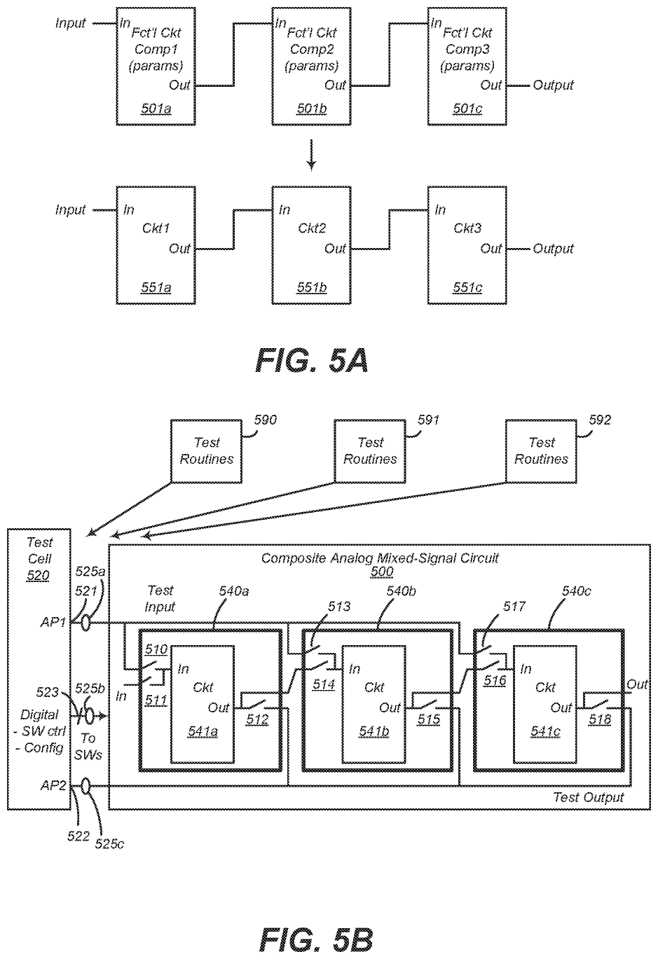

FIGS. 5A-B illustrate an example of circuitry generated in non-DFT and DFT mode according to various embodiments. In this example, three configurable functional circuit components 501a-c are coupled together in series (e.g., in a user interface of a behavioral simulator). Each component 501a-c may have different parameters that are set (e.g., by a user) to achieve the desired functionality of the composite circuit, for example. Here, an analog input is received by component 501a. An output of component 501a is coupled to an input of component 501b, and an output of component 501b is coupled to an input of component 501c. In this illustrative example, the output of the combined circuit is taken at the output of component 501c.

Components 501a-c may be converted to transistor level analog or mixed signal circuit modules 551a-c. In a non-DFT mode, the input of circuit module 551a and the output of circuit module 551c may be accessible, but intermediate inputs and outputs, as well as internal nodes in each circuit, may not be accessible when all the circuit modules are coupled together, for example. For an integrated circuit produced from modules 551a-c, testing would be from the “outside-in”, where AC and DC voltages and currents may be applied and sensed at the input of 551a and output of 551c to determine functionality of the composite circuit.

Advantageously, embodiments of the present disclosure include one or more test modes where a range of different circuitry may be added to one or more circuit modules to allow AC or DC voltages or currents to be applied or sensed from a variety of internal nodes, such as intermediate nodes between circuit modules or interior nodes of particular circuit modules, for example. Various test modes may result in a range of different testing options (e.g., selectable by a user) to allow more or fewer test points and test options across a range of different test and verification applications. Selectively allowing users to turn DFT on and off for different components of a design advantageously provides flexibility in achieving test scan coverage across a design while minimizing the additional circuitry (and cost) required to achieve it.

FIG. 5B illustrates an example transistor level schematic EDA environment including test cell 520 and a composite circuit 500 comprising circuit modules 540a-c. In this example, DFT has been turned on for components 501a-c. Accordingly, generator software selects sub-circuit schematics and DFT sub-circuit schematics to produce circuit modules 540a-c. In this illustrative example, the same non-DFT sub-circuits schematics may be used to produce circuits 541a-c as above in FIG. 5A. However, additional sub-circuit schematics for switch circuits 510-518 are also included (e.g., in the circuit modules 540a-c) to allow DFT test cell 520 independent access to input and output nodes for circuit modules 540a-c, for example, on a test input line and a test output line. In this example, the input of circuit module 540a is coupled to an input of circuit 500 through switch 511 (closed during normal operation) and to a test input through switch 510. Similarly, an output of circuit module 540a is coupled to an input of module 540b through switch 514 (closed during normal operation) and to a test output through switch 512. Accordingly, during a test mode, switches 510 and 512 can be closed and the other switches open. In that state, an input signal (voltage or current) may be applied to the input of test circuit module 540a on the test input and an output measured on the test output. Switches 513/514/515 and 516/517/518 are similarly arranged to directly and independently test circuit modules 540b and 540c, respectively. It is to be understood that, in various example implementations, other inputs or outputs or internal analog nodes of any circuit module may be selectively accessed using the switch circuits and one or more test input and test output connections. In some example embodiments, one or more of switches 510/511, 513/514 and 516/517 may be implemented as analog multiplexers mentioned above or using the circuits shown in FIG. 4C, for example.

This example further includes test cell 520. As mentioned above, test generator software may select particular test routines, such as test routines 590-592 for testing circuit modules 540a-c, respectively. Test routines 590-592 may be combined and converted into test cell 520 for testing the composite analog or mixed signal circuit comprising circuit modules 540a-c, for example. Test cells according to various embodiments may include test routines used for DFT off and/or DFT on modes, for example, based on which functional circuit components in the chip design have DFT turned on and off.

In this simple illustrative example, test cell 520 may include two (2) analog test pins (AP1, AP2, sometimes referred to herein as “analog test terminals”) 521/522 coupled to pins 525a/525c of circuit 500. Further, digital signals 523 of test cell 520 are coupled to pins 525b (e.g., a digital bus) of circuit 500 to interface with circuits 540a-c. Example analog test pins may generate (e.g., force) and/or measure analog signals, such as a direct current (DC) analog voltage, DC analog current, alternating current (AC) analog voltage (e.g., a waveform), or an AC current, for example. An AP may be able to force a voltage, force a current, measure a voltage, or measure a current, for example. AP1 521 may force input voltage signals (AC or DC) and AP2 522 may be configured to measure output voltage signals from circuits 541a-c, for example. Digital signals 523 may open and close switches 510-518 during different tests and/or may be used to configure each circuit module 540a-c. For example, digital signals 523 may open and close switches and/or enable (e.g., power on or off) different circuit modules 540a-c independently under control of a test script executed when test cell 520 is run in the EDA environment to test circuit 500.

As an example, during a first test, all switches except switches 510 and 512 may be open, circuit module 540a is enabled and circuit modules 540b-c are disabled, and AP1 may generate an analog AC or DC voltage to the input of circuit 541a. AP2 may sense an AC or DC voltage or current on the output of circuit 541a through switch 512 and perform a measurement, for example. During another test, all switches except switch 513 and 515 may be open and circuit module 540b is enabled while the other circuit modules are disabled. In this case, AP1 and AP2 may test circuit module 541b. Similarly, during another test, all switches except switches 517 and 518 are open and circuit module 541c may be tested. During normal operation (non-test), switches 511, 514, and 516 may be closed and the other switches open so that the circuit may be configured in the same way as circuit modules 551a-c in FIG. 5A, for example.

In some embodiments, AP1 and AP2 may be set to perform a different set of tests on circuits 541a-c, for example. One advantage of the present technique of sharing APs in a transistor level schematic across multiple analog/mixed signal circuit modules is that numerous circuit modules can be tested with fewer numbers of AP test interfaces on a semiconductor chip, for example. It should be noted that in some example embodiments the test cell 520, circuit modules 541a-c, and switches 510-518 are transistor level schematics (e.g., in an EDA environment, such as Cadence® or Mentor®). Accordingly, the analog voltages and currents to and from the APs, as well as the voltages and currents in the circuit modules may be simulated (as mentioned above in connection with FIG. 3A). One advantage of certain embodiments of the present disclosure is that the transistor level schematics for the circuit modules 541a-c, switches 510-518, and digital lines 523 may be automatically incorporated into a layout of an integrated circuit and used to manufacture a semiconductor chip having an integrated circuit including the circuit modules 541a-c, switches 510-518, and digital lines 523 described above. Automated test equipment (e.g., lab bench equipment or a production ATE tester) may connect actual analog voltage/current generators, voltage/current measurement units, and digital signals between test interface 525a-c and test inputs/outputs of the chip (e.g., pads on a semiconductor die or pins on a packaged die) and run some or all of the same test routines to test the actual physical semiconductor devices that were used to verify the transistor level schematics, for example. Thus, advantageously, either or both of an EDA verification test system or an ATE test program may be generated automatically from the selected test routines to verify both the transistor level schematics and the physical integrated circuit, for example.

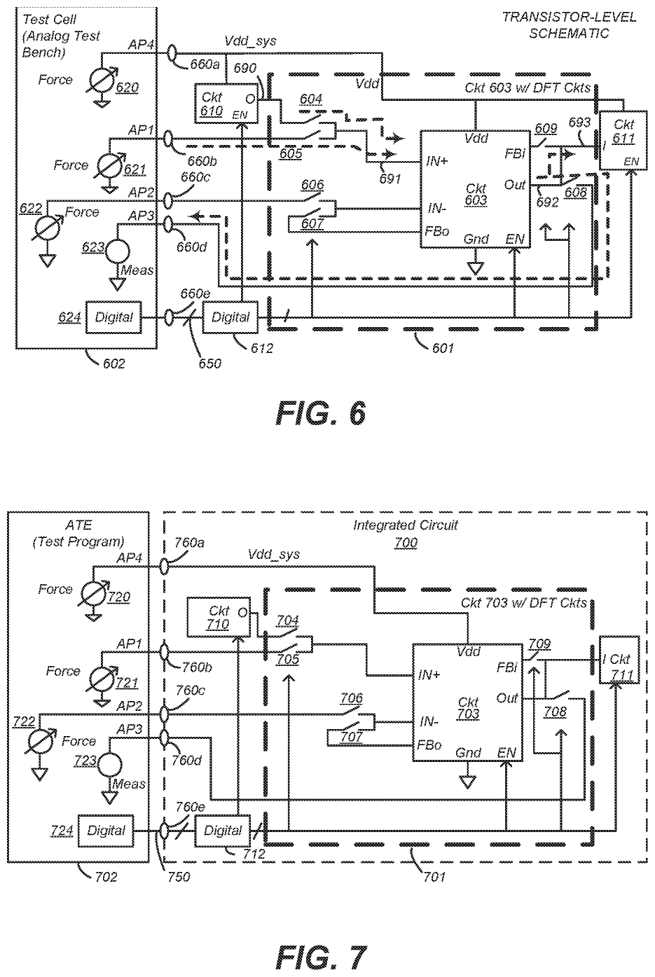

Features and advantages of the present disclosure may further include automatically converting test routines for particular functional circuit components (e.g., embedded in a chip design) to both transistor level schematic verification test cells and ATE test programs, which advantageously reduces development time and cost to manufacture an analog or mixed signal integrated circuit. The following examples shown in FIGS. 6-7 illustrate testing an embedded parameterized circuit module schematic 601 and physical circuit 701 across an EDA and physical test environments from a common test routine. Connections of circuit modules 610/710 and 611/711 to the test interface are omitted in these examples but are illustrated in examples below.

FIG. 6 illustrates transistor level schematic verification according to an embodiment. In this example, a test cell 602 (aka, an analog test bench) and multiple circuit modules 610, 601, and 611 (each of which may be generated from parameterized functional circuit components) are included in a transistor level schematic in an EDA system. Test cell 602 has been converted to HDL executable in the EDA system from a test routine for the functional circuit component. An embedded circuit module 601 is coupled to a test cell 602.

Test cell 602 may be generated from test routines as described above. In this example, circuit module 601 is configured between circuit modules 610 and 611 corresponding to other functional circuit components having particular parameters, for example. A plurality of switch circuits 604-609 may be configured to selectively couple analog signals between analog nodes of circuits 610, 603, and 611 in a normal operating mode (e.g., along a signal path from circuit 610 to circuit 603 to circuit 611), and between analog nodes and analog test terminals (APs) during a test mode. For instance, during normal operation, an analog signal may be coupled from an analog output node (O) 690 of circuit 610 to an analog input node (IN+) 691 of circuit 603, and another analog signal may be coupled from an analog output node (OUT) 692 of circuit 603 to an analog input node (I) 693 of circuit 611. During testing, module 603 may be configured so that analog input node 691 and analog output node 692 are coupled to test cell 602.

More specifically, switches 604 and 605 have terminals coupled together and coupled to analog input node 691. Switch 604 has a terminal coupled to analog output node 690 of circuit 610. Switch 605 has a terminal coupled to test interface 660b and analog test terminal, AP1, which may be coupled to an analog source 621, for example. Source 621 may be configured to generate a simulated voltage or current as specified in the corresponding test routine used to generate test cell 602. At the output of circuit 603, switch 608 selectively couples and decouples analog output node 692 to test interface node 660d and AP3, which may be coupled to an analog measurement unit 623 (e.g., a probe), for example. Measurement unit 623 may be configured to measure a simulated output voltage or current generated during a simulation of circuit 601 in response to a simulated input from AP′, for example. Some nodes, such as output node 692, may only use 1 switch to selectively coupled and decouple to an analog test terminal. In this case, node 692 is coupled to an analog input node (I) 693 of circuit module 611, which may be high impedance, and therefore may not need to be decoupled from output node 692 during test, for example. Alternatively, module 611 may include switches to decouple from node 692. In other embodiments, other nodes may use pairs of switches, such as switches 604/605 and 606/607, so that one signal path may be disconnected while the other is connected (e.g., during testing and normal operation as mentioned above). Accordingly, in some embodiments, the switch circuits, such as pairs of switches 604/605 and 606/607, may be analog multiplexers, for example.

As mentioned above, test routines may be converted into test cells, and the test cells may be configured to perform a plurality of tests on a circuit schematic, such as circuit schematic including module 601. The tests may generate simulated voltages and/or currents on analog test terminals of the test cell 602, and the simulated voltages/currents are simulated through transistor level circuit schematic 601 to produce simulated outputs. The simulated outputs, in turn, are coupled from analog output nodes of circuit schematic 601 to an analog test terminal of test cell 602. The simulated test results may be displayed to a user, for example, to determine if circuit 601 is operating as desired. A variety of tests may be performed by test cell 602 on circuit 601.

FIG. 6 illustrates another feature of some example embodiments. In this example, a digital circuit 612 is generated and configured between digital test interface 660e and one or more circuit modules (e.g., here, circuit modules 610, 601, and 611). For example, test cell 602 may comprise a plurality of digital connections (aka lines) 650 coupled to digital circuit 612. Test cell 602 may include a digital routines 624 for sending digital signals on digital lines 650. Digital circuit 612 may translate digital signals on digital lines 650 into digital control signals for opening and closing switches in the circuit modules, controlling one or more multiplexers, enabling circuit modules or sub-circuits of the modules, or otherwise configuring the circuit modules for normal operation or testing, for example. For example, digital circuit 612 may have individual wires coupled to each switch 604-609, for example, for opening and closing each switch. Digital circuit 612 may include one or more wires for sending digital control signals to configure circuit modules 601, 610, and/or 611. In this example, digital circuit 612 includes a wire coupled to an enable (EN) digital input for turning circuit 603 on and off. In some embodiments, digital connections 650 may be a bus (e.g., an I²C bus) and multiple digital circuits 612 have different addresses on the bus. Accordingly, a plurality of digital circuits 612 may be accessed and used to configure one or more circuit modules (e.g., 601, 610, 611) into a test mode, and the circuit modules may be tested independently through a test interface comprising analog test interface terminals (e.g., analog interface terminals 660b-d) and digital test interface terminals (e.g., digital test interface terminals 660e).

One example test illustrated here is a supply current test. In this example, circuit 601 may include a power supply analog input node Vdd coupled to a system power supply, Vdd_sys, for receiving a power supply voltage during normal operation. Vdd_sys may be coupled to a terminal or pad of the integrated circuit (i.e., a chip pad). In a simulator, terminal 660a may be coupled to a simulated power supply 620 (here, AP4), for example. Accordingly, during a power supply test, AP1 may be configured to generate a power supply voltage and measure the current, for example, to test power supply current. In this example, Vdd_sys is also coupled to circuit modules 610 and 611. Advantageously, in various embodiments, one or more of the circuit modules may be independently turned on and off (e.g., shutdown using an enable pin). In this example, digital signals 650 may cause digital controller 612 to shutdown circuit modules 610 and 611 (and any other circuits drawing current from Vdd_sys) during a supply current test, for example, so that only circuit module 601 is drawing current during the test.

As another example, during normal operation analog input node IN− of circuit 601 may be coupled through switch 607 to internal feedback output node (FBo) and switch 609 may couple an output signal to an internal feedback input node (FBi), for example. During a test, switches 604, 607 and 609 may be opened and switches 605, 606, and 608 may be closed. AP1 and AP2 may apply voltages to the inputs to perform open loop testing, for example, which may be measured by AP3, for example. Accordingly, test cell 602 may configure and reconfigure switches and/or circuit 601 during different time periods to perform a variety of different tests. It is to be understood that the particular tests and switch configurations illustrated here are merely examples, and that a wide range of other test circuit configurations for different circuit modules and different tests may be used. It is to be understood that the analog sources (e.g., sources 620-622), measurement units (e.g., unit 623), and digital unit (e.g., unit 624) may comprise HDL code, such as Verilog-AMS or Verilog-A, which may be controlled as specified in the above described test routines to operate according to a variety of conditions when executed in a simulation of an EDA environment, for example.

FIG. 7 illustrates integrated circuit testing according to an embodiment. Advantageously, in some embodiments, an integrated circuit 700 produced from the schematic of FIG. 6 may be coupled to computer controlled automated test equipment (ATE) and tested using a program derived from the same test routines used to generate test cell 602. In this example, an ATE test program executable ATE 702 was generated from the same test routine for the functional circuit component used to generate test cell 602. The same tests, for example, may be applied to a transistor level circuit schematic 601 in a simulator and to the corresponding integrated circuit 700 by ATE 702. ATE 702 may include a digital bus 724 (e.g., I²C) including bus wires 750 for interfacing with integrated circuit 700 over a digital interface 760e. Integrated circuit 700 may have one or more digital circuits 712 for configuring interconnected circuit modules 710, 711, and 701 (including switches 704-709). Accordingly, ATE 702 may execute a test program to test circuit 701 in substantially the same way that test cell 602 tested the transistor level schematic version of the circuit module 601. Accordingly, selected test routines may be converted to an ATE program and executed on an ATE test system (e.g., either an automated bench test system or production test system). The ATE may verify that the physical integrated circuit operates as simulated, for example. Thus, some embodiments of the present disclosure automatically generate both transistor level schematic verification test cell and ATE programs, which advantageously reduces development time and cost to manufacture an analog or mixed signal integrated circuit.

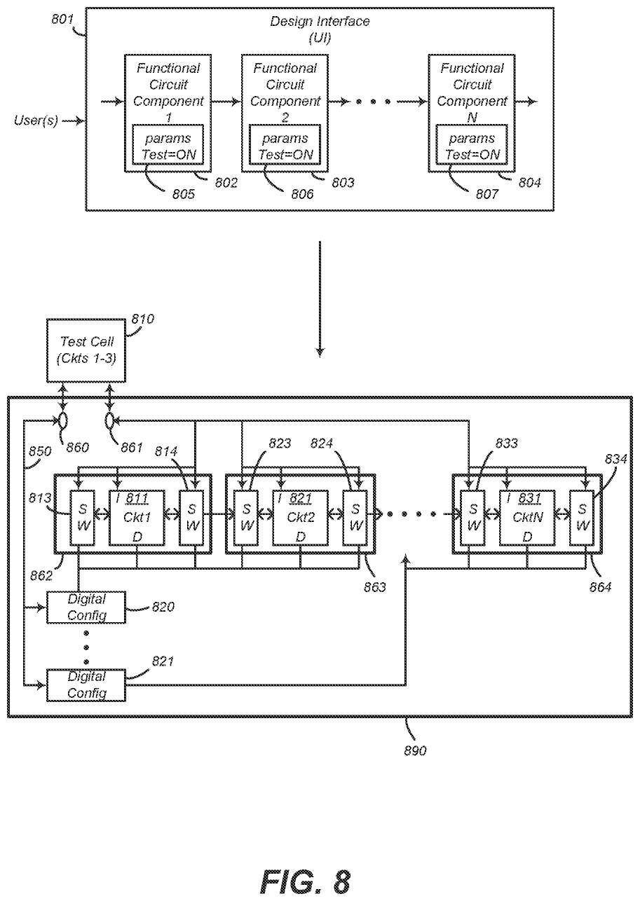

FIG. 8 illustrates a verification system for parameterized functional circuit components and corresponding circuit modules according to another embodiment. In this example, a user has specified N (an integer) functional circuit components 802-804 in a user interface (UI) 801 with corresponding parameter values 805-807, respectively, and turned DFT on for each one. While the components are illustrated here as in series, it is to be understood that a wide variety of other circuit configurations known to those skilled in the art may be used. The parameter values may be used to select test routines and DFT sub-circuits (and non-DFT sub-circuits) to generate test cell 810 and transistor level schematics for N circuit modules denoted here as 862, 863, and 864 corresponding to the N parameterized functional circuit components 802, 803, and 804, respectively, for example. Test cell 810 is coupled to circuit 890 (here, in an EDA environment) via one or more test interface terminals 861 and a plurality of digital configuration interface terminals 860. Test interface terminals 861 may comprise one or more analog input terminals, one or more analog output terminals, one or more digital input terminals, and one or more digital output terminals, for example. Digital configuration interface terminals 860 may be a bus (e.g., I2C) comprising multiple digital lines 850 for configuring one or more modules in circuit 890, for example.

In this example, analog voltage or current may be coupled to inputs and/or outputs of operational circuitry 811, 821, and 831 in circuit modules 862, 863, and 864 through switches 813-814, 823-824, and 833-834, for example. Analog voltage or current may also be coupled to or from internal nodes (I) in core circuitry 811, 821, and 831 of each circuit module 862-864. Additional switches or multiplexers may be used for internal nodes (not shown). Advantageously, circuit modules 862, 863, and 864 may be independently configured and tested in response to digital signals received on digital bus 850, which is coupled to a plurality of addressable digital configuration circuits 820-821. For example, digital configuration circuit 820 may be sent a digital message (e.g., address and instructions), which may turn on particular digital wires coupled to particular analog switches, enable inputs, or other digitally controlled analog circuits in a particular module (e.g., digital inputs “D” on module 862). A digital configuration circuit (e.g., 820 or 821) may support configuration and control of one, part of one, or multiple analog circuit modules 862-864, depending on how many digital wires or control inputs each module may have, for example. In this example, digital configuration circuit 820 supports configuration and testing of circuit modules 862 and 863. Other digital configuration circuits may support one particular circuit module, for example. For circuit modules with many configurations, multiple digital configuration circuits may be used.

While all of the functional circuit components 802, 803, and 804 and corresponding circuit modules 862, 863, and 864 are illustrated here as having DFT turned on, it is to be understood that in other embodiments some of the components and corresponding modules may have DFT turned off. In applications where a portion of the components have DFT turned on and other components have DFT turned off, verification of the components having DFT turned off may be performed by coupling signals from components with DFT turned on into the components with DFT turned off, and then sensing the behavior in yet other components with DFT turned on, for example. For three components configured in series, for example, if the first and last components have DFT turned on, then testing of the middle component, which has DFT turned off, may be performed by sending signals to the first component and sensing signals in the last component, for example.

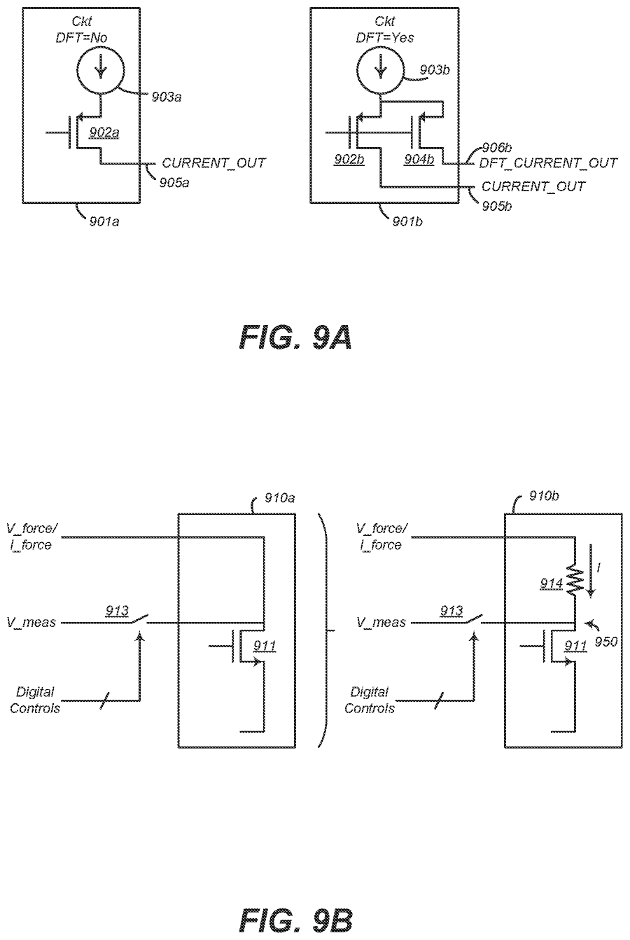

FIG. 9A illustrates accessing internal nodes of a circuit according to an embodiment. In this example, when DFT is off, parameter values may be used to select a sub-circuit 901a comprising a current source 903a coupled through a transistor 902a to a current out pin 905a. Sub-circuit 901a may be combined with other circuits to form a circuit module corresponding to a functional circuit component, for example. However, when DFT is turned on, an alternative sub-circuit 901b may be selected. Sub-circuit 901b comprises a current source 903b and transistor 902b, which are configured in substantially the same way as in sub-circuit 901a to produce a current on pin 905b. Additionally, sub-circuit 901b includes a current sense transistor 904b having an auxiliary current output (e.g., which may be coupled to an internal node of sub-circuit 901b or directly accessible to measure current). Transistor 904b may be configured to produce a current on pin 906b (e.g., DFT CURRENT OUT) that is related to the current through transistor 902b (e.g., equal to or a fraction of). In this example, a gate of transistor 904b is coupled to a gate of transistor 902b and a source of transistor 904b is configured to receive current from current source 903b. Accordingly, pin 906b may be coupled to an analog test terminal (e.g., through an analog switch) in an EDA environment or on an ATE to measure the current in transistor 904b and verify the current in transistor 902b, for example.

FIG. 9B illustrates accessing internal nodes of a circuit according to another embodiment. In this example, a sub-circuit 910a includes a power switch comprising a transistor 911 that may be sized to pass a large amount of current. When DFT is on, sub-circuit 910a may include switch 913 to test the power switch (e.g., when DFT is off, the switch may not be included in the circuit). Switch 913 may be controlled by digital wires from an addressable digital configuration circuit described above, for example. A voltage source may force voltage and measures current, for example, on a terminal of a transistor (here, the drain). Switch 913 may selectively couple a sense line to an analog test output to measure voltage, for example. As illustrated in equivalent circuit 910b, wires connected to certain internal nodes (e.g., the drain of power switch 911) may include resistive voltage drops (illustrated here as resistor 914). Accordingly, by sensing an internal node (e.g., here, node 950 at the drain of transistor 911), a more precise voltage on the node may be obtained. Forcing voltage to a node on one pin along a current carrying path and measuring voltage on another pin that does not carry the primary current is sometimes referred to as a Kelvin connection. Accordingly, embodiments of the present disclosure may advantageously allow Kelvin connections to be established on internal nodes of circuit modules for use in testing, for example.

It is to be understood that the techniques illustrated in FIGS. 9A-B are merely illustrative examples of a wide variety additional circuitry that may be added to couple internal nodes of an analog circuit module to one or more externally accessible test interface pins when DFT is turned on for the circuit module. A wide variety of other circuits may be used to selectively couple voltages and currents from particular analog circuit modules to one or more test analog output lines shared by multiple circuit modules on an integrated circuit, for example.

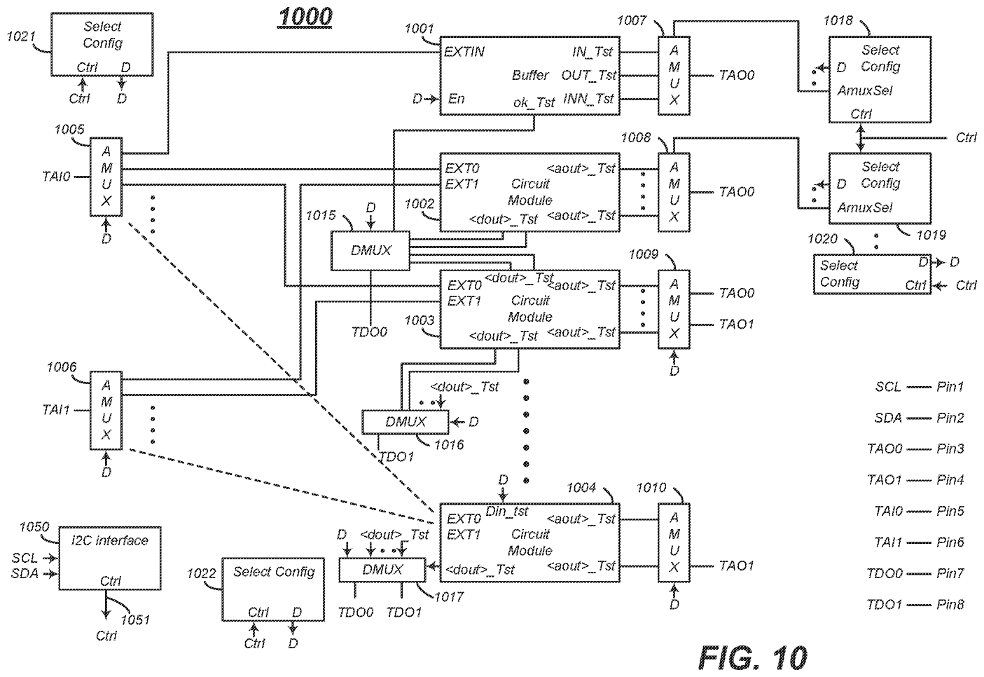

FIG. 10 illustrates example test circuitry on an analog/mixed signal integrated circuit 1000 according to an embodiment. The example shown in FIG. 10 illustrates a plurality of circuit modules 1001-1004 corresponding to parameterized functional circuit components, which may be interconnected to perform certain functions during normal operation. Normal operational interconnections are not shown here. Rather, FIG. 10 illustrates how circuit modules may be arranged with additional circuits to form a test architecture according to certain embodiments. For instance, in some embodiments, an integrated circuit 1000 may include one or more analog test terminals (e.g., wafer pads or device pins) selectively coupled to a plurality of circuit modules through a plurality of switch circuits (e.g., analog multiplexers). For example, analog test input terminals TAI0 and TADI1 may selectively couple analog signals (e.g., AC or DC voltages or currents) from an external source (e.g., off chip) to the integrated circuit and to a plurality of circuit modules (e.g., as voltage or current inputs). Conversely, analog test output terminals TAO0 and TAO1 may selectively couple analog signals (e.g., AC or DC voltages or currents) from a plurality of circuit modules (e.g., as voltage or current outputs) to an external measurement system (e.g., off chip), for example.

This example illustrates a buffer circuit module 1001 configured on an integrated circuit with a plurality of other circuit modules 1002-1004. The circuit modules may be coupled together to perform a particular function during normal operation. The example in FIG. 10 shows the configuration of the test circuitry. For example, buffer 1001 includes an external analog input (EXTIN), a digital test output ok_Tst, and analog test outputs IN_Tst, OUT_Tst, and INN_Tst, as described above in connection with FIG. 4C, for example. In this example, the analog test inputs terminals (e.g., TAI0, TAI1) may be coupled to a switch circuit, such as an analog multiplexer (AMUX) 1005, and the output of the switch circuit is coupled to various analog inputs of circuit modules 1001-1004 (e.g., EXTIN of buffer 1001). Similarly, the analog test outputs of circuit modules 1001-1004 may be coupled to a switch circuit, such as an analog multiplexer (AMUX) 1007, and the output of the switch circuit may be coupled to one of a plurality of analog test output terminals (e.g., TAO0 and/or TAO1). A switch circuit comprises one or more transistors configured to receive a first signal on one terminal and a control signal on a control terminal (e.g., a gate). In response to the control signal state, the one or more transistors selectively couple the first signal to an output terminal of the switch circuit (e.g., by turning one or more transistors on or off). Analog multiplexers (AMUX) are an example of a switch circuit that may be used in various embodiments.

Here, analog outputs from buffer 1001 are coupled through AMUX 1007 to a first analog test output terminal TAO0. Analog outputs from circuit module 1004 are coupled through AMUX 1010 to a second analog test output terminal. Analog outputs from circuit module 1003 may be coupled through AMUX 1009, for example, to either or both of analog test output terminals TAO0 and/or TAO1. Accordingly, analog test outputs of a circuit module may be selectively coupled to one analog test output TAO0 (as in the case for buffer 1001) or to multiple analog test output terminals (as in the case for circuit module 1003). As illustrated here, multiple analog circuit nodes of the same module or different modules may be coupled to the same test analog output terminal to advantageously allow testing of particular features of particular modules inside the integrated circuit. Accordingly, analog outputs of the same or different circuit modules may share the analog test output terminals by performing different tests at different time periods and reconfiguring the AMUXs between tests, as needed, for example. In some cases, multiple analog test output terminals (e.g., TAO0 and TAO1) may advantageously allow multiple analog signals to be measured from the same or different circuit modules simultaneously, for example. Thus, an integrated circuit may be advantageously tested from the inside out.

Similarly, circuit modules 1001-1004 may generate digital test outputs <dout>_tst. Such digital test outputs may be coupled through switch circuits such as digital multiplexers (DMUXs) 1015-1017, for example, to one or more digital test output terminals, e.g., TDO0 and TDO1. Digital test output terminals shared across one or more circuit modules may allow for determine operability of subcircuits in the modules using, for example, a variety of built-in self-test circuits that generate a positive digital signal when the test is passed (e.g., successful power up or a variety of self-checking circuits on various subcircuits in the module, for example).

The switch circuits may be controlled by a plurality of configurations circuits. For example, here, AMUXs 1005-1010 coupled to TAI0, TAI1, TAO0 and/or TAO1 and DMUXs 1015-1017 are selected using a plurality of select configuration circuits 1018-1022. Each select configuration circuit 1018-1022 may be configured over a control bus. For example, each select configuration circuit 1018-1022 may have a different address (e.g., a unique address on the particular integrated circuit). Each select configuration circuit 1018-1022 may further have logic to set one or more data bits (D) high or low based on signals received over the bus. For example, select configuration circuit 1018 may have one or more digital bit lines (D) (here, AmuxSel) coupled to AMUX 1007 to select which test analog outputs of buffer 1001 are coupled to TAO0. If select configuration circuit 1018 receives a signal on control bus (Ctrl) that specifies the address for configuration circuit 1018 and an instruction to set AmuxSel bit(s), then the AmuxSel bit(s) are set according to the bus instruction and AMUX 1007 is configured to pass the desired analog signal. Accordingly, instructions on a control bus (Ctrl) may be used to configure the AMUXs and DMUXs and control the routing of the test analog input signals on TAI0 and TAI1, control the routing of the test analog output signal on TAO0 and TAO1, and/or control the routing of the test digital output signals TDO0 and TDO1, for example. In some embodiments, circuit modules may have digital inputs for configuring the operation of the circuit module. In one embodiment, a select configuration circuit has one or more digital bit outputs (D) couple to a digital input of a circuit module (e.g., the enable input of buffer 1001). Circuit module 1004 may have a test digital input, Din tst, coupled to one or more digital data inputs (D) of a select configuration circuit. Accordingly, the digital values of Din tst may be set on the fly over the control bus (Ctrl) to change the behavior of a circuit module and/or provide test digital inputs for testing the circuit module, for example.

Select configuration circuits 1018-1022 may be controlled over the control bus, which may also be coupled an interface controller digital circuit. In this example, select configuration circuits 1018-1022 are configured over control bus (Ctrl) by an I2C interface circuit 1050. In this example, I2C interface 1050 may include a serial data terminal, SDA, and a serial clock terminal, SCL. Accordingly, an external system may communicate with integrated circuit 1000 to configure one or more specific circuit modules for testing. Integrated circuit 1000 may have a plurality of pins, such as Pins 1-8, where Pins 1 and 2 are used to communicate with the I2C interface 1050 (over clock SCL and data SDA lines), Pins 3 and 4 are coupled to the test analog output terminals TAO0 and TAO1, Pines 5 and 6 are coupled to the test analog input terminals TAI0 and TAI1, and Pins 7 and 8 are coupled to the test digital output terminals TDO0 and TDO1, respectively.

As mentioned above, switch circuits for coupling analog and digital signals between test terminals of the integrated circuit and test nodes of particular circuit modules may be configured using select configuration circuits that receive data over an internal test configuration and control bus (Ctrl) 1051. For example, when a particular configuration circuit detects it's address on the control bus (Ctrl) line, it translates the instruction on the bus into output commands (e.g., digital signals to select a particular MUX path or to enable or otherwise configure a digital input of a circuit module). It is to be understood that a variety of control bus architectures could be used in various embodiments. Additionally, embodiments of the present disclosure may further automatically generate the instance of a synchronous bus interface circuit 1050 (e.g., a 2-wire clock and data bus such as I2C (i-squared-c)).

Advantageously, analog test input signals may be generated off chip and coupled to inputs of particular circuit modules (including internal nodes) through analog test input terminals, and analog test outputs from the particular circuit modules (including outputs generated by internal nodes) may be coupled to analog test output terminals for measurement by an off chip measurement system. These techniques may advantageously improve the ability to determine where a failure is occurring in a circuit, isolate the location of a malfunctioning circuit, or obtain performance data for particular parameterized functional circuit components thereby reducing the time it takes to design analog/mixed signal semiconductor circuits, for example.

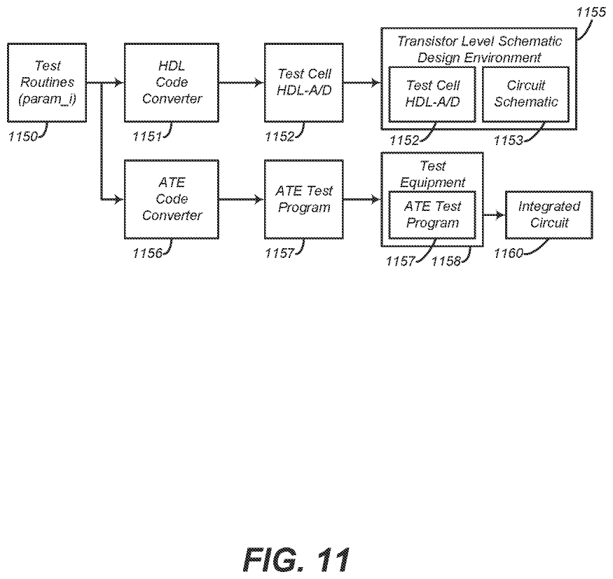

As illustrated above, features and advantages of some embodiments may use test routines to test various embodiments of functional circuit components, including, for example, schematic representations of functional circuit components, physical semiconductor embodiments of functional circuit components (e.g., integrated circuits), or both, for example. FIG. 11 illustrates an example of how test routines may be applied to various embodiments of functional circuit components. In this example, test routines 1150 are received by a code converter (or translator) 1151. The test routines 1150 may have been selected based on, among other criteria, particular parameter values (“param_i”) as described above, for example.

As discussed above in connection with FIG. 1B, code converter 1151 may convert test routines 1150 into test cells 1152, which may be code in a hardware description language for describing analog and digital circuits (“HDL-A/D”) such as VHDL or Verilog versions including analog circuits (e.g., Verilog A or AMS). Test cells 1152 may be imported into an electronic design automation (EDA) system 1155, such as Cadence® or Mentor®, for example, and used to verify the functionality of a transistor level circuit schematic 1153. As illustrated above, test cells 1152 may be used to test and verify a circuit schematic 1153 comprising a plurality of circuit modules generated from a corresponding plurality of functional circuit components each having its own parameters and parameter values, for example.

Advantageously, in some embodiments, test routines 1150 may be used to automatically generate test systems for either or both of a transistor level schematic verification and/or a physical semiconductor device verification. For example, in some embodiments, an ATE code converter 1156 may receive test routines 1150 and generate an automated test program 1157. For example, test routines 1150 may be converted into a test program 1157, which may be executed by automated test equipment (ATE). As mentioned above, when the test routines 1150 are selected based on particular parameter values (e.g., param_i) that are also used to generate a transistor level schematic of a circuit including a number of parameterized functional circuit components, such test routines 1150 may be automatically converted into an ATE test program 1157 for testing an integrated circuit chip embodying the same parameterized functional circuit components. Automated test equipment (ATE) 1158 may include a wide range of systems, but may specifically include voltage/current sources, voltage/current meters, waveform generators (aka function generators), and waveform measurement systems. In some embodiments, the ATE test program 1157 is used to control benchtop test equipment (e.g., a bench setup) comprising one or more voltage/current sources, one or more voltage/current meters, one or more waveform generators, and one or more waveform measurement systems (e.g., an oscilloscope), where the test program may be executed by a computer coupled to the test equipment over a bus for controlling the test equipment to generate and/or measure voltage, current, frequency, or other electrical characteristics, for example. In other embodiments, test equipment 1158 may be a semiconductor ATE for analog or mixed signal production test of integrated circuits as are well known to those skilled in the art, including systems with analog or mixed signal testing capability offered by Teradyne®, Advantest®, or Cohu®, for example. Such systems may load ATE test program 1157 into a control unit, and execution of the test program issues command signals to a variety of test system components (e.g., test head cards) for generating voltage/current, measuring voltage/current, generating waveforms, and/or measuring waveforms, for example. Accordingly, some embodiments of the present disclosure may automatically select test routines that may be used to generate transistor level verification and/or physical verification (e.g., automated bench testing and/or production testing), for example.

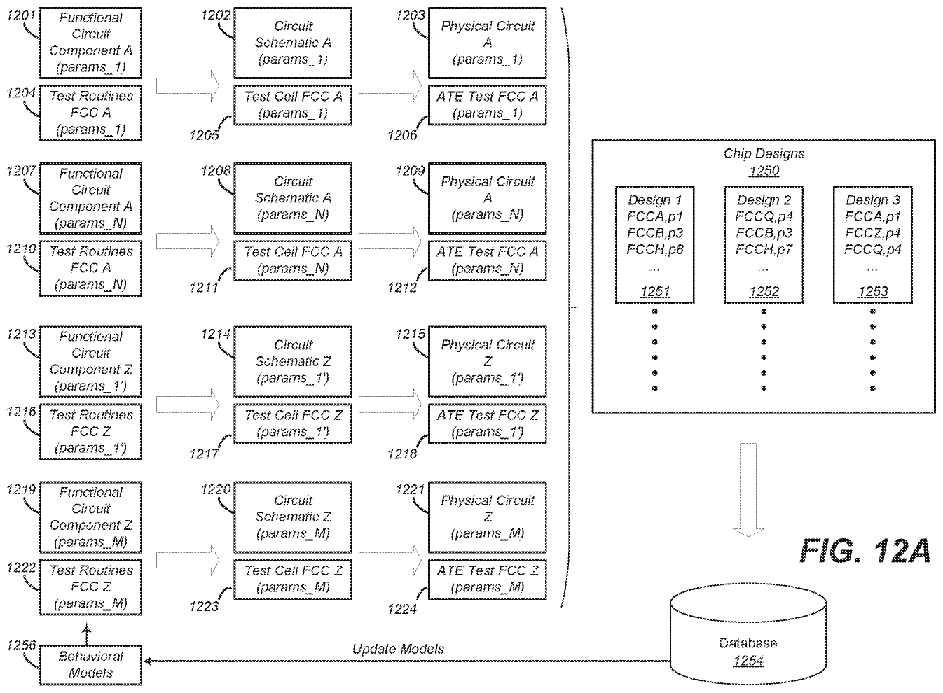

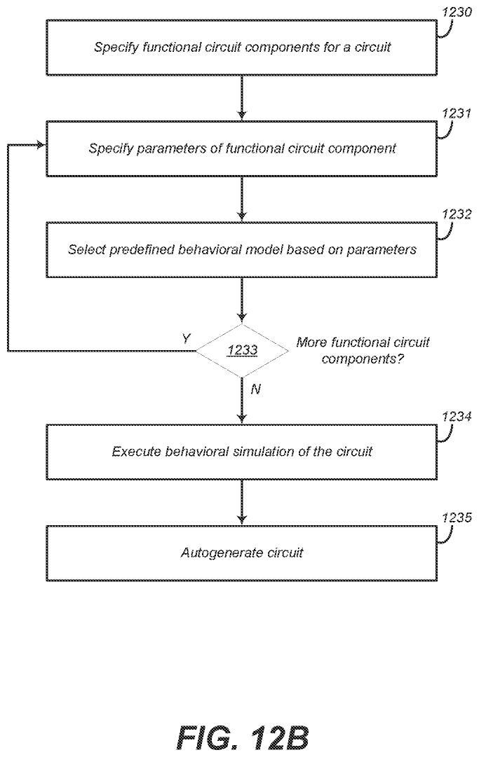

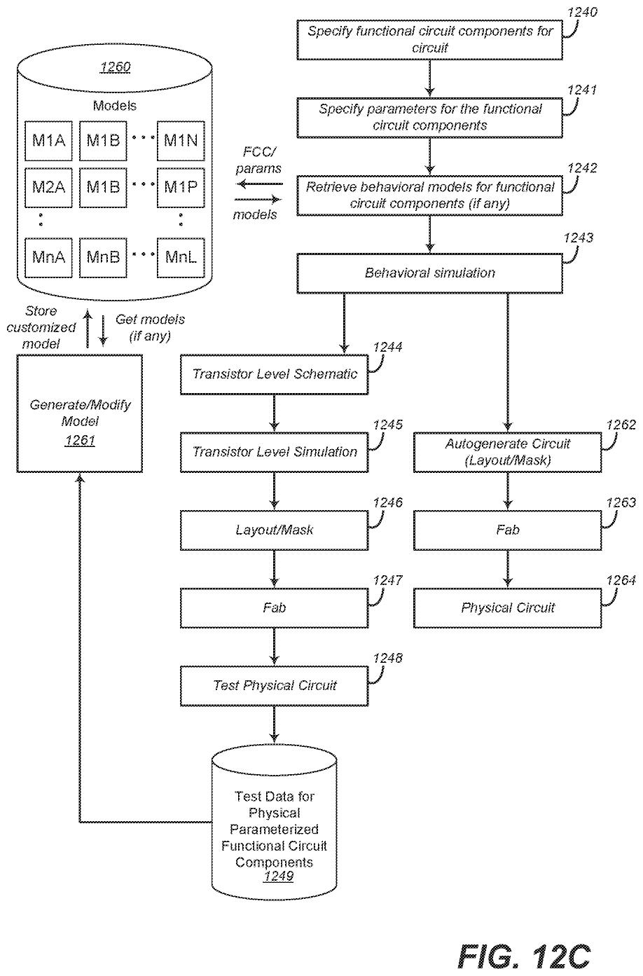

FIG. 12A illustrates testing and data collection according to another embodiment. In some cases, it may be desirable to gather performance data for functional circuit components to characterize the behavior of the functional circuit components across all their different parameters. Since some embodiments allow testing of functional circuit components embedded inside different designs, characterization/operational data may be acquired for each functional circuit component across different circuit designs that the functional circuit component is used in. Such data, obtain over time and across multiple circuit designs, may be stored in a database and used to refine behavioral models for each functional circuit component.

FIG. 12A illustrates a plurality of functional circuit components (FCCA-FCCZ), each configurable with parameters. In this example, FCCA may be configured with N (an integer) different parameters (e.g., FCCA 1201 has parameter values “params_1” and FCCA 1207 has parameter values “params_N”). Similarly, FCCZ may be configured with M (an integer) different parameters (e.g., FCCZ 1213 has parameter values “params_1′” and FCCA 1207 has parameter values “params_M”). As mentioned above, each set of parameter values for each FCC may be used to select test routines, such as test routines 1204 for FCCA:params_1, test routines 1210 for FCCA:params_N, test routines 1216 for FCCZ:params_1′, and test routines 1222 for FCCZ:params_M. The various parameterized functional circuit components and corresponding test routines may be used in various chip designs where they are converted to circuit schematics and test cells (schematics 1202, 1208, 1214, and 1220 and corresponding test cells 1205, 1211, 1217, and 1223, respectively). The schematics may, in turn, be converted to layouts to produces physical circuits, and the test routines may be converted to corresponding ATE test programs (e.g., physical circuits 1203, 1209, 1215, and 1221 and corresponding ATE test programs 1206, 1212, 1218, and 1224).

The various parameterizations of the FCCs may be incorporated into chip designs as illustrated at 1250. Different designs may use different FCCs having different parameters, and some designs will have the same FCCs with the same parameters. In this example, design 1251 has different FCCs with different parameters than designs 1252 and 1253, but design 1252 uses FCCA with the same parameters (p1) as design 1253. Since FCCs may be tested while embedded in a plurality of designs using the at least some of the same tests derived from the same test routines, data for the same parameterized functional circuit components may be obtained across designs, for example, to improve the characterization of the functional circuit component. In this example, test data for all the parameterized FCCs are stored in a database 1254. The test data may include a plurality of test results for different tests The test data may include data distributions for particular tests for each FCC having the same parameters (e.g., the same gain) or tests that span multiple parameterizations (e.g., offset voltage). Advantageously, test data describing the behavior of each physical manifestation of each functional circuit component may be used to update behavioral models 1256 used to model each functional circuit component, for example, thereby increasing the accuracy of the behavioral models used to design the integrated circuits.

As mentioned above, embodiments of the present disclosure may include behavioral models generated from data specifying the physical behavior of functional circuit components (aka, test data or characterization data). As illustrated below, some embodiments may be behavioral models for functional circuit components having particular parameters. Accordingly, the parameters may be used to select predefined behavioral models that may be very accurate (e.g., models generated from test data of physical functional circuit components with those parameters). Accordingly, behavioral simulations of the circuit may be sufficiently accurate to move straight to mask generation, while skipping traditional and computationally intensive transistor level simulations, for example. For example, behavioral simulations of a full top-level circuit (e.g., an entire analog/mixed signal integrated circuit) may be significantly faster and less computationally intensive than a transistor level simulation of the full top-level circuit.

FIG. 12B illustrates generating a circuit according to an embodiment. At 1230, a plurality of functional circuit components are specified for a circuit to be generated. In various embodiments, functional circuit components may be parameterized building blocks of larger circuit, where the parameters change the characteristics of the functional circuit component. In some embodiments the functional circuit components are analog functional circuit components for generating analog circuits. For instance, functional circuit components may be one or more of: a comparator circuit, an oscillator circuit, a delay circuit, a current generator circuit, a voltage reference circuit, an amplifier circuit, a voltage buffer circuit, a bandgap circuit, a current mirror circuit, a transconductance circuit, and a voltage-to-current converter circuit. In some example embodiments, functional circuit components have a plurality of corresponding parameters, where different values for each of the plurality of corresponding parameters produce functional circuit components with different characteristics. Accordingly, at 1231, parameters are specified for each functional circuit component. Functional circuit components for a circuit to be generated may be specified in a variety of ways. For example, in one embodiment, a user may drag graphical representations of the functional circuit components into a circuit design canvas and enter the parameter values for each functional circuit component to form the circuit. In other embodiments, a user may input a specification for a circuit to be generated, and a software engine (e.g., using predefined rules) may select the required functional circuit components and configure the parameters for each functional circuit component to form the circuit. At 1232, predefined behavioral models are selected based on the parameters. For instance, different parameters for a same functional circuit component (e.g., two values for amplifier gain) result in selection of different corresponding behavioral models (e.g., one model optimized for each gain value). As described in more detail below, behavioral models according to the present disclosure may be optimized for particular functional circuit component parameters. In one example, a behavioral model is customized to accurately model a functional circuit component for one or more parameters values (e.g., dominant parameters, such as gain or offset). In other examples, a behavioral model is customized for each unique set of parameters. For example, if an amplifier's parameters are gain, offset, and input type, a different behavioral model for each unique set of parameter values may be generated and stored for use when an amplifier having those unique set of parameter values is part of a circuit design. At 1233, steps 1231 and 1232 are repeated for any additional functional circuit components in the circuit to be generated. At 1234, a behavioral simulation of the circuit to be generated may be executed based on the selected predefined behavior models. Advantageously, because the behavioral models are generated based on data collected from actual silicon circuits of the functional circuit components having the particular specified values, they are sufficiently accurate to move to generation of the circuit at 1235 (e.g., without transistor level simulation). For example, generation of the circuit at 1235 includes automatically generating the circuit schematic based on the parameterized functional circuit components and generating the mask for the circuit. Advantageously, the transistor level schematics and mask are generated based on the behavioral simulation of the circuit and not a transistor level simulation of the circuit, for example.

As illustrated further below, the presently described technique is possible because, for example, numerous predefined behavioral models corresponding to particular functional circuit components having different user specified parameters are generated from test data, which is gathered from one or more automatically generated circuits comprising the functional circuit components with the particular parameters. In some cases, a functional circuit component (e.g., a comparator) with particular parameters may be on a single circuit, and test data from the circuit (e.g., including the comparator with particular parameters) may be used to model the functional circuit component when the same component and same parameters are used again in the future. In other cases, the functional circuit component with particular parameters may be on multiple different circuits, and test data from the circuits includes particular data for the functional circuit component with particular parameters. Accordingly, data from multiple circuits may include data characterizing the behavior of the same functional circuit component with the same parameters, which can be used to model the functional circuit component when the same component and same parameters are used again in the future. Thus, test data from multiple different circuits may be disaggregated and categorized by functional circuit component and the functional circuit components particular values. Data for the same component with the same parameters across multiple different integrated circuits is combined to improve the modeling of the component having the particular parameters over time (e.g., and dominant parameters may be identified).

FIG. 12C illustrates generating a circuit according to another embodiment. In a first phase of the process, one or more circuits are generated from parameterized functional circuit components using traditional transistor level simulation techniques, converted to masks, fabricated, and tested. Data from working functional circuit components with particular parameter values are used to generate custom behavioral models. In a second phase, circuits comprising functional circuit components with corresponding custom behavioral models are advantageously simulated using the behavioral model, and masks are generated without the need for additional time-consuming transistor level simulations.

For example, during a design phase, functional circuit components are specified at 1240. At 1241, the parameters for the functional circuit components are specified. At 1242, behavioral models for the functional circuit components are retrieve from a storage location 1260 (if they exist). For example, initially, a functional circuit component may have a generic behavioral model used for any and all parameter values. Accordingly, when a behavioral simulation is performed at 1243, the results may not match the actual results in an actual physical (e.g., silicon) implementation. At 1244, a transistor level schematic of the circuit to be generated is produced (e.g., automatically by software) and simulated at 1245 to verify the functionality of the functional circuit components and the circuit as a whole (e.g., top level functionality). The transistor level functional circuit components and/or top level circuit may be adjusted as necessary to obtain satisfactory performance. Software for generating the functional circuit components and the circuit as a whole may be modified accordingly. At 1246, the transistor level circuit is converted to a mask (e.g., automatically by software). At 1247, an integrated circuit is generated in a fabrication facility based on the mask. At 1248, the physical circuit is tested to obtain data specifying an actual behavior of the functional circuit components having the specified parameters in the fabricated circuit. Data on particular functional circuit components may be obtained through a variety of techniques, including some of the techniques described above. Test data from multiple physical circuits each comprising multiple functional circuit components having the same or different parameter values may be stored in a data repository 1249. For each functional circuit component, a customized behavioral model corresponding to the functional circuit component having the particular parameters is generated at 1261 based on at least a portion of the data (e.g., a data for a comparator with particular parameters, data for a current mirror having particular parameters, data for an amplifier having particular parameters). If a generic behavioral model already existed for a functional circuit component, then the generic behavioral model may be modified to produce a customized behavioral model. The result is that functional circuit components with particular parameter values will each have a corresponding predefined behavioral model customized to fit the test data from a physical implementation of that functional circuit component and particular parameter values. Over time, in some embodiments, the same functional circuit components with the same parameters may be reused and embedded in multiple integrated circuits. Therefore, the predefined behavioral models are modified based on the test data for those components to generate the customized behavioral models corresponding to particular parameters.

For example, different parameter values for a comparator functional circuit component may produce N (where N is an integer greater than 1) different types of comparators and N different transistor level schematics of comparators with different electrical characteristics. After layout and fabrication, each of the N comparators may be tested. Testing may involve collecting data for a wide variety of electrical characteristics as are known to those skilled in the art. Test data may include multiple data points for each of the electrical characteristics taken across many instances of each of the N comparators. For example, fabrication may produce M (where M is an integer greater than 1) physical comparator die having the same parameters. Thus, test data may be gathered for M×N physical circuits, where M is the number of physical comparator circuits having the same parameters and N is the number of different types of comparators. Each test on each of the M×N physical comparators may be repeated numerous times to produce many data points for each particular electrical characteristic. Statistical analysis may be performed on the data to determine averages, means, standard deviations, and the like for each of the N different types of comparators, for example. Curves with many data points may be produced from test data. Test data may include a wide range of data commonly referred to as characterization data, which characterizes the behavior of a circuit. Characterization data for each of the N comparators may be generated. Curve fitting algorithms may be used to build behavioral models of the N different types of comparators. In some embodiments, behavioral models may comprise piecewise linear models describing the behavior of the comparator. Accordingly, for N different types of comparators derived from the comparator functional circuit component parameters, there may be N different unique and independent behavioral models, for example, which may be selected and used to perform a behavioral simulation of a circuit. The same techniques may be applied to other functional circuit components, for example.

The second phase of the process may occur when a design uses functional circuit components and parameters with corresponding behavioral models available in storage 1260. Functional circuit components for another circuit to be generated are specified at 1240. Typically, different groups of functional circuit components are used in different circuit designs, but the same group of functional circuit components may be used with the same or different parameter values, for example. Parameters are specified at 1241. At 1242, predefined behavioral models corresponding to the functional circuit component with the particular specified parameters (aka, predefined parameterized behavioral models) are selected and retrieved from storage 1260. As illustrated here, a particular functional circuit component may have a plurality of behavioral models based on the parameters. For example, models M1 may all be associated with a particular functional circuit component (e.g., an amplifier), where model M1A is associated with the amplifier having a first set of parameter values, model M1B is associated with the amplifier having a second set of parameter values, and so on up to model M1N, where N is an integer. Similarly, another functional circuit component may have a plurality of behavioral models based on the parameters. For example, models M2 may all be associated with another particular functional circuit component (e.g., a comparator), where model M2A is associated with the comparator having a first set of parameter values, model M2B is associated with the comparator having a second set of parameter values, and so on up to model M2P, where P is an integer. Different functional circuit components may have different numbers of parameters and different numbers of parameter values. Accordingly, different functional circuit components may have different numbers of customized behavioral models. Thus, different parameters for a same functional circuit component result in selection of different corresponding predefined parameterized behavioral models. Once the customized behavioral models for the functional circuit components in the circuit are retrieved, behavioral simulations may be executed at 1243. A behavioral simulation of the circuit to be generated is execute at 1262 based on the predefined parameterized behavior models for the plurality of functional circuit components. Advantageously, because the behavioral models are optimized and customized for particular parameters of each functional circuit component based on actual integrated circuit test data, the simulations are more accurate. Accordingly, at 1262 the circuit and may be generated based on a behavioral simulation of the circuit and not a transistor level simulation of the circuit, for example. At 1263, the circuit is fabricated, and a physical circuit is obtained at 1264.

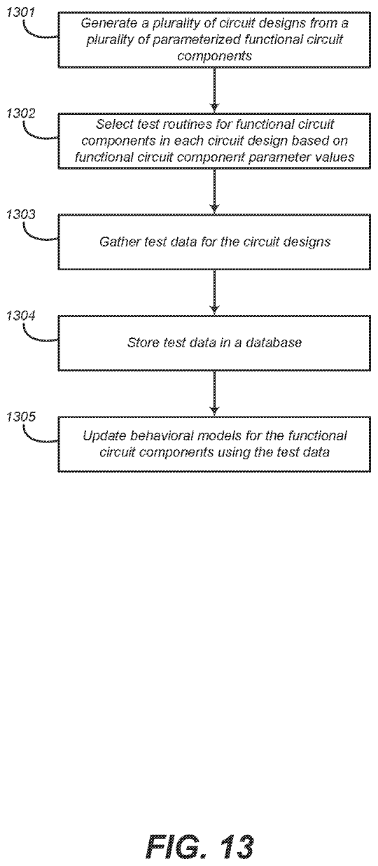

FIG. 13 illustrates a computer implemented method according to an embodiment. At 1301, a plurality of circuit designs may be generated from a plurality of functional circuit components. The functional circuit components may comprise a plurality of parameters, where the circuit designs have at least some different circuit modules corresponding to different functional circuit components having different parameters. A user may perform a behavioral simulation of the circuit design. Once the behavioral simulation works as desired (e.g., once the required functional circuit components are included in the design and set with the correct parameter values), the user may trigger generator software to generate a transistor level schematic for the design (and a layout). At 1302, a plurality of test routines are selected for functional circuit components in the circuit designs. The test routines may be selected based, at least in part, on parameter values of the functional circuit components in the circuit design. At 1303, a plurality of test data are gathered for the plurality of circuit designs. The test data may comprise results of a plurality of tests for each functional circuit component, for example. At 1304, the plurality of test data is stored in a database. At 1305, the behavioral models for the functional circuit components are updated using the test data.

### Example Computer Systems

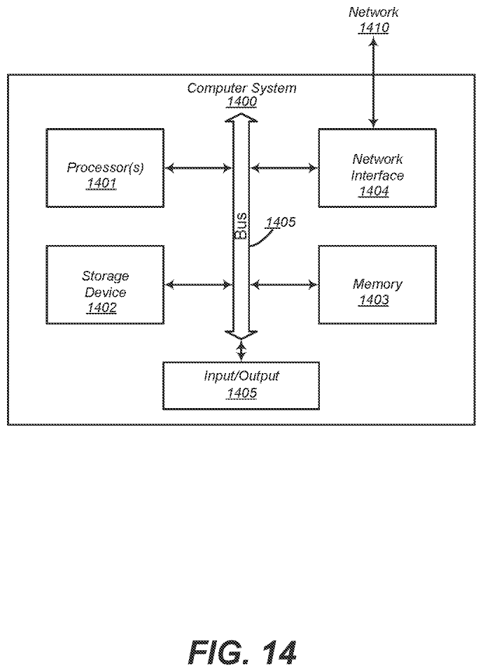

FIG. 14 illustrates example computer system hardware according to certain embodiments. Various embodiments of the present disclosure may be implemented in computer executable code (software) for performing various techniques described herein. FIG. 14 illustrates a simplified example of a computer used to execute software according to various embodiments. One or more such computers may be used to perform the techniques described herein. As shown in FIG. 14, a computer system 1400 may include one or more processors 1401, such as a multi-processor CPU (e.g., from Intel® or AMD®). Processors 1401 may load executable program code from a computer readable storage medium, which when executed, performs some or all of the techniques described herein. Processors 1401 may be coupled to one or more buses, which are represented here as bus 1405, to communicate with various subsystems. In this illustrative example, processor(s) 1401 may communicate with a storage device 1402 (e.g., to retrieve executable code), which may comprise magnetic storage, optical storage, or solid-state storage, for example. Processor(s) 1401 may further communicate with memory 1403 over a bus 1405. Memory 1403 may represent a number of memories including a random-access memory (RAM) for storage of instructions and data during program execution and a read-only memory (ROM) 1420 in which fixed instructions are stored, for example. In some embodiments, processor(s) 1401 may communicate with removable media (e.g., CD-ROM, DVD, Blu-Ray, etc.), a removable flash memory-based drive or card, and/or other types of storage media known in the art.

Processor(s) 1401 may communicate with a network interface 1404 over a bus 1405, for example. Network interface 1404 may allow computer system 1400 to communicate with a network 1410 which may be in communication with other computer systems to perform the techniques described herein, for example. In various embodiments, network interface 1404 may include, for example, an Ethernet card to support a variety of Ethernet rate connections (e.g., in an office, server room, or data center), a Wi-Fi, IEEE 802 based system, and/or cellular adapter, a modem (telephone, satellite, cable, ISDN, etc.), digital subscriber line (DSL) units, and/or other data communications systems, for example.

In some embodiments, the techniques described herein may be performed on one or more server computers. In other embodiments, some or all of the techniques described herein may be performed on a laptop or desktop computer or other computer system that may include an input/output interface 1405, for example. Input/output interface 1405 may include hardware for receiving information from an input device (e.g., a mouse, keyboard, touchpad, or the like) and/or provide information to an output device (e.g., a display).

Although bus 1405 is represented here as a single bus, it is to be understood that bus 1405 may comprise multiple busses.

It will be appreciated by those skilled in the art that computer system 1400 is illustrative and many other configurations having more or fewer components than system 1400 are possible.

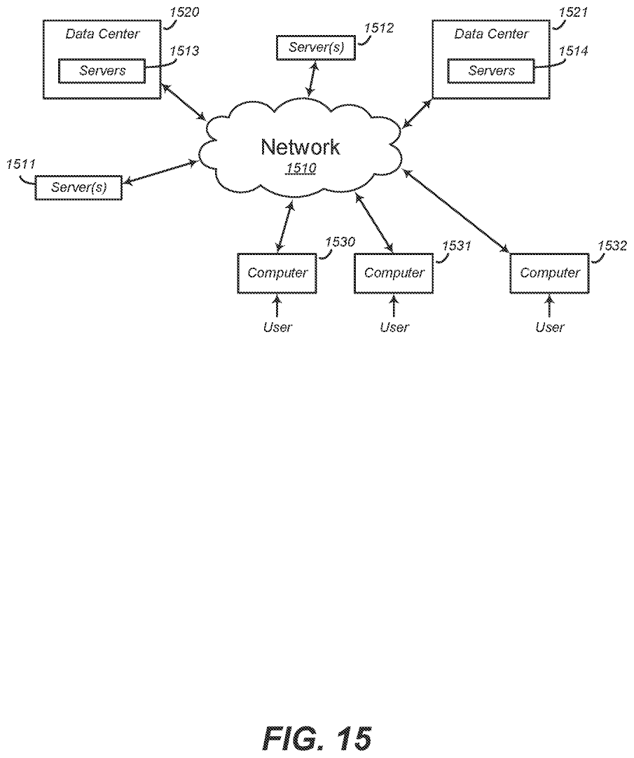

In some embodiments, features and techniques described herein may be embodied in software executing on remote computer systems (e.g., software as a service executing in the cloud). FIG. 15 illustrates various computer system configurations that may be used in certain embodiments. In this example, the executable code for performing the some or all of techniques disclosed herein may be executing on one or more server computers coupled together over a network 1510, such as a local area network (LAN), wide area network (WAN), or other networks (e.g., the Internet). Computer executable code for performing some or all of the techniques disclosed herein may be executed on a single server or multiple servers 1511-1513, for example. Servers 1511-1513 may have computer architectures as described in FIG. 15. Software according to various embodiments may be executed directly by a server or be run on a virtual machine, which executes on computer system hardware, for example, as known to those skilled in the art. In some embodiments, embodiments of the disclosure may be performed on one or more servers that are part of data centers 1520-1521, for example.

As mentioned above, some embodiments described herein may receive inputs from users. Accordingly, users may interact with computer systems 1530-1532, which in turn communicate with one or more server computers 1511-1514 over network 1510 to perform the techniques described herein, for example.

### Further Example Embodiments

Each of the following non-limiting examples may stand on its own or may be combined in various permutations or combinations with one or more of the other examples.

In one embodiment, the present disclosure includes a computer-implemented method comprising: receiving, by at least one software system executing on at least one computer, information specifying a plurality of circuit specification parameters corresponding to at least one analog functional circuit component; and selecting, based on the circuit specification parameters, a subset of test routines from a plurality of test routines for the at least one analog functional circuit component, wherein the plurality of test routines are configured to test the at least one analog functional circuit component across different values of the circuit specification parameters.

In one embodiment, a first subset of test routines is selected from a first plurality of test routines when a first functional circuit component has a first set of parameter values, and wherein a second subset of test routines is selected from the first plurality of test routines when the first functional circuit component has a second set of parameter values.

In one embodiment, a plurality of test routines associated with different parameter values have different associated test conditions.

In one embodiment, a plurality of test routines associated with different parameter values have different associated test usages.

In one embodiment, the test usages comprise one or more of: a production test usage, a characterization usage, and a typical test usage.

In one embodiment, a first functional circuit component type has a first set of parameters and a first plurality of test routines, and a second functional circuit component type has a second set of parameters and a second plurality of test routines.

In one embodiment, the method further comprises converting selected test routines to test cells, wherein the test cells are executable by a transistor level schematic simulator.

In one embodiment, the method further comprises converting selected test routines to test programs executable by an automated test system.

In one embodiment, the method further comprises selecting, based on the circuit specification parameters, a plurality of analog sub-circuit schematics corresponding to the at least one analog functional circuit component to produce at least one transistor level circuit module schematic.

In one embodiment, the method further comprises selecting, based on at least one circuit specification parameter value, at least one analog test sub-circuit schematic, wherein the at least one transistor level circuit module schematic includes said at least one analog test sub-circuit schematic.

In one embodiment, the analog test sub-circuit schematic comprises a plurality of switch circuits.

In one embodiment, different combinations of analog sub-circuit schematics for a particular functional circuit component having different circuit specification parameter values are tested using different subsets of test routines.

In one embodiment, the method further comprises generating one or more test cells based on the selected subset of test routines, wherein the one or more test cells are coupled to the at least one transistor level circuit module schematic.

In one embodiment, the method further comprises simulating the test cells and the transistor level circuit module schematic together to test the functionality of the circuit module schematic.

In one embodiment, the test cells comprise hardware description language code.

In one embodiment, the hardware description language code is one of Verilog-AMS or Verilog-A.

In one embodiment, the transistor level circuit module schematic comprises a plurality of switch circuit schematics configured between internal nodes of the circuit module schematic and the one or more test cells.

In one embodiment, the plurality of switch circuit schematics form at least one analog multiplexer.

In one embodiment, at least one switch circuit schematic couples an analog signal between an analog node of the circuit module schematic and an analog test terminal of the one or more test cells in a test mode, and wherein the at least one switch circuit schematic decouples the analog node of the circuit module schematic from the analog test terminal of the one or more test circuit schematics in a non-test mode.

In one embodiment, the method further comprises generating a plurality of switch circuit schematics configured between analog nodes of the circuit module schematic and analog nodes of other circuit module schematics corresponding to one or more other functional circuit components.

In one embodiment, at least one switch circuit schematic decouples an analog signal between a first analog node of the circuit module schematic and a second analog node of another circuit module schematic during testing, and wherein the at least one switch circuit schematic couples the analog signal between the first analog node of the circuit module schematic and the second analog node of said another circuit module schematic during normal operation.

In one embodiment, a plurality of subsets of test routines are selected for a plurality of functional circuit components.

In one embodiment, each functional circuit component is tested by a corresponding subset of test routines independently of other functional circuit components.

In one embodiment, each subset of test routines is associated with instructions for turning on a corresponding functional circuit component and turning off one or more other functional circuit components.

In another embodiment, the present disclosure includes and integrated circuit comprising: a plurality of circuit modules corresponding to analog functional circuit components, the plurality of circuit modules comprising analog circuits; and a plurality of switch circuits, the plurality of switch circuits comprising: a first plurality of switch circuits configured between inputs of corresponding circuit modules and outputs of preceding circuit modules; a second plurality of switch circuits configured between outputs of the corresponding circuit modules and inputs of the subsequent circuit modules; a third plurality of switch circuits configured between the inputs of the corresponding circuit modules and at least one analog test input; and a fourth plurality of switch circuits configured between analog nodes of the corresponding circuit modules and at least one analog test output; and one or more digital configuration circuits comprising: a plurality of digital inputs configured to receive digital signals from an external source; and a plurality of digital outputs coupled to the plurality of switch circuits, wherein, during normal operation, the first plurality of switch circuits and second plurality of switch circuits are closed and the third plurality of switch circuits and fourth plurality of switch circuits are open, and wherein, during testing, the first plurality of switch circuits and second plurality of switch circuits are open and the third plurality of switch circuits and fourth plurality of switch circuits are closed.

In one embodiment, during testing, the at least one analog test input is coupled to an external source and the at least one analog test output is couple to a measurement sy stem.

In one embodiment, one or more of the analog nodes are an internal nodes.

In one embodiment, one or more of the analog nodes are outputs of the corresponding circuit modules.

In one embodiment, the plurality of switch circuits further comprising a fifth plurality of switch circuits, the fifth plurality of switch circuits configured between internal analog nodes of the corresponding circuit modules and the at least one analog test output.

In one embodiment, the third plurality of switch circuits are selectively coupled to the at least one analog test input through a sixth plurality of switch circuits.

In one embodiment, the sixth plurality of switch circuits form one or more analog multiplexers.

In one embodiment, the sixth plurality of switch circuits form an analog tree structure between the at least one analog test input and the third plurality of switch circuits.

In one embodiment, the fourth plurality of switch circuits are selectively coupled to the at least one analog test output through a sixth plurality of switch circuits.

In one embodiment, the sixth plurality of switch circuits form one or more analog multiplexers.

In one embodiment, the sixth plurality of switch circuits form an analog tree structure between the fourth plurality of switch circuits the at least one analog test output.

In one embodiment, at least a portion of the switch circuits coupled to each circuit module are configured by one or more corresponding addressable digital configuration circuits.

In one embodiment, the digital configuration circuits each comprise an address, and wherein switch circuits of the plurality of switch circuits coupled to a first circuit module are opened and closed in response to one or more corresponding digital configuration circuits receiving a first address and data.

In one embodiment, the digital configuration circuits are coupled to a synchronous bus interface circuit to receive data from an external source.

In another embodiment, the present disclosure includes computer-implemented method comprising: receiving, by at least one software system executing on at least one computer, parameters corresponding to at least one analog functional circuit component the parameters comprising an activated test mode parameter; and selecting, based on the circuit specification parameters and the activated test mode parameter, a plurality of subcircuit schematics, wherein the subcircuit schematics include circuitry for externally accessing the at least one functional circuit component when the at least one functional circuit component is embedded in a circuit design; and generating a transistor level schematic for the circuit design, the transistor level schematic comprising circuitry for accessing one or more of an input node, an output node, or an internal node of the at least one analog functional circuit component.

In one embodiment, a different plurality of subcircuit schematics are selected when the test mode parameter is deactivated.

In one embodiment, the integrated further comprises selecting a subset of test routines from a plurality of test routines for the at least one analog functional circuit component, wherein the plurality of test routines are configured to test the at least one analog functional circuit component across different values of the circuit specification parameters.

In another embodiment, the present disclosure includes a computer-implemented method comprising: generating a plurality of circuit designs from a plurality of functional circuit components, the functional circuit components comprising a plurality of parameters, wherein the circuit designs have at least some different circuit modules corresponding to different functional circuit components having different parameters; selecting, for the functional circuit components in the circuit designs, a plurality of test routines, wherein the test routines are selected based, at least in part, on parameter values of the functional circuit component parameters; gathering a plurality of test data for the plurality of circuit designs, the test data comprising results of a plurality of tests for each functional circuit component; storing the plurality of test data in a database; and updating behavioral models for the functional circuit components using the test data.

In another embodiment, the present disclosure includes a computer-implemented method comprising: specifying a plurality of functional circuit components for a circuit to be generated; for each of one or more of the functional circuit components of the plurality of functional circuit components: specifying parameters of the functional circuit component; and selecting, based on the parameters, a predefined behavioral model corresponding to the functional circuit component having the specified parameters, wherein different parameters for a same functional circuit component result in selection of different corresponding behavioral models; executing a behavioral simulation of the circuit to be generated based on the selected predefined behavior models for the plurality of functional circuit components.

In one embodiment, the method further comprises generating a mask for the circuit, wherein the mask is generated based on the behavioral simulation of the circuit and not a transistor level simulation of the circuit.

In one embodiment, a plurality of predefined behavioral models corresponding to particular functional circuit components having different user specified parameters are generated from characterization data from one or more automatically generated circuits comprising said functional circuit components.

In one embodiment, the method further comprises receiving data specifying the physical behavior of at least one of the plurality of functional circuit components having particular specified parameters; and modifying one or more behavioral models corresponding to particular functional circuit components having particular specified parameters based on at least a portion of the data.

In one embodiment, the parameters are generated in response to user inputs.

In one embodiment, the functional circuit components are analog functional circuit components for generating analog circuits.

In one embodiment, the functional circuit components have a plurality of corresponding parameters, wherein different values for each of the plurality of corresponding parameters produce functional circuit components with different characteristics.

In one embodiment, the functional circuit components are one or more of: a comparator circuit, an oscillator circuit, a delay circuit, a current generator circuit, a voltage reference circuit, an amplifier circuit, a voltage buffer circuit, a bandgap circuit, a current mirror circuit, a transconductance circuit, and a voltage-to-current converter circuit.

In another embodiment, the present disclosure includes a method comprising: specifying a plurality of functional circuit components for a circuit to be generated; specifying parameters of the plurality of functional circuit components; and generating a mask for the circuit to be generated comprising the plurality of functional circuit components having the specified parameters; fabricating the circuit based on the mask; storing data specifying an actual behavior of the plurality of functional circuit components having the specified parameters in the fabricated circuit; and for each functional circuit component, generating a customized behavioral model corresponding to the functional circuit component having the specified parameters based on at least a portion of the data.

In one embodiment, the plurality of functional circuit components each having corresponding predefined behavioral models, and wherein the predefined behavioral models are modified based on said at least a portion of the data to generate the customized behavioral models corresponding to the specified parameters.

In one embodiment, the predefined behavioral models are modified based on at least a portion of the data to generate customized behavioral models corresponding to unique combinations of parameters.

In one embodiment, the method further comprises specifying a second plurality of functional circuit components for a second circuit to be generated; for each of one or more of the second plurality of functional circuit components: specifying parameters of a particular functional circuit component; selecting, based on the parameters, a particular predefined parameterized behavioral model corresponding to the functional circuit component having the specified parameters, wherein different parameters for a same functional circuit component result in selection of different corresponding predefined parameterized behavioral models; and executing a behavioral simulation of the second circuit to be generated based on one or more predefined parameterized behavior models for the plurality of functional circuit components.

In one embodiment, the method further comprises generating a mask for the second circuit, wherein the mask is generated based on a behavioral simulation of the circuit and not a transistor level simulation of the circuit.

In one embodiment, the parameters are user specified.

In one embodiment, the functional circuit components are analog functional circuit components for generating analog circuits.

In one embodiment, the functional circuit components have a plurality of corresponding parameters, wherein different values for each of the plurality of corresponding parameters produce functional circuit components with different characteristics.

In one embodiment, the functional circuit components are one or more of: a comparator circuit, an oscillator circuit, a delay circuit, a current generator circuit, a voltage reference circuit, an amplifier circuit, a voltage buffer circuit, a bandgap circuit, a current mirror circuit, a transconductance circuit, and a voltage-to-current converter circuit.

In one embodiment, the data is test data for the circuit representing an actual behavior of the circuit.

The above description illustrates various embodiments of the present disclosure along with examples of how aspects of the particular embodiments may be implemented. The above examples should not be deemed to be the only embodiments, and are presented to illustrate the flexibility and advantages of the particular embodiments as defined by the following claims. Based on the above disclosure and the following claims, other arrangements, embodiments, implementations and equivalents may be employed without departing from the scope of the present disclosure as defined by the claims.

What is claimed is:

1. A computer-implemented method comprising:
receiving, by at least one software system executing on at least one computer, information specifying a plurality of circuit specification parameters corresponding to at least one analog functional circuit component; and
selecting, based on the circuit specification parameters, a subset of test routines from a plurality of test routines for the at least one analog functional circuit component, wherein the plurality of test routines are configured to test the at least one analog functional circuit component across different values of the circuit specification parameters.

2. The method of claim 1 wherein a first subset of test routines is selected from a first plurality of test routines when a first functional circuit component has a first set of parameter values, and wherein a second subset of test routines is selected from the first plurality of test routines when the first functional circuit component has a second set of parameter values.

3. The method of claim 1 wherein a plurality of test routines associated with different parameter values have different associated test conditions.

4. The method of claim 1 wherein a plurality of test routines associated with different parameter values have different associated test usages.

5. The method of claim 4 wherein the test usages comprise one or more of: a production test usage, a characterization usage, and a typical test usage.

6. The method of claim 1 wherein a first functional circuit component type has a first set of parameters and a first plurality of test routines, and a second functional circuit component type has a second set of parameters and a second plurality of test routines.

7. The method of claim 1 further comprising converting selected test routines to test cells, wherein the test cells are executable by a transistor level schematic simulator.

8. The method of claim 1 further comprising converting selected test routines to test programs executable by an automated test system.

9. The method of claim 1 further comprising selecting, based on the circuit specification parameters, a plurality of analog sub-circuit schematics corresponding to the at least one analog functional circuit component to produce at least one transistor level circuit module schematic.

10. The method of claim 9 further comprising selecting, based on at least one circuit specification parameter value, at least one analog test sub-circuit schematic, wherein the at least one transistor level circuit module schematic includes said at least one analog test sub-circuit schematic.

11. The method of claim 10 wherein the analog test sub-circuit schematic comprises a plurality of switch circuits.

12. The method of claim 9 wherein different combinations of analog sub-circuit schematics for a particular functional circuit component having different circuit specification parameter values are tested using different subsets of test routines.

13. The method of claim 9 further comprising generating one or more test cells based on the selected subset of test routines, wherein the one or more test cells are coupled to the at least one transistor level circuit module schematic.

14. The method of claim 13 further comprising simulating the test cells and the transistor level circuit module schematic together to test the functionality of the circuit module schematic.

15. The method of claim 13 wherein the test cells comprise hardware description language code.

16. The method of claim 15 wherein the hardware description language code is one of Verilog-AMS or Verilog-A.

17. The method of claim 13 wherein the transistor level circuit module schematic comprises a plurality of switch circuit schematics configured between internal nodes of the circuit module schematic and the one or more test cells.

18. The method of claim 17 wherein the plurality of switch circuit schematics form at least one analog multiplexer.

19. The method of claim 17 wherein at least one switch circuit schematic couples an analog signal between an analog node of the circuit module schematic and an analog test terminal of the one or more test cells in a test mode, and wherein the at least one switch circuit schematic decouples the analog node of the circuit module schematic from the analog test terminal of the one or more test circuit schematics in a non-test mode.

20. The method of claim 17 further comprising generating a plurality of switch circuit schematics configured between analog nodes of the circuit module schematic and analog nodes of other circuit module schematics corresponding to one or more other functional circuit components.

21. The method of claim 20 wherein at least one switch circuit schematic decouples an analog signal between a first analog node of the circuit module schematic and a second analog node of another circuit module schematic during testing, and wherein the at least one switch circuit schematic couples the analog signal between the first analog node of the circuit module schematic and the second analog node of said another circuit module schematic during normal operation.

22. The method of claim 1 wherein a plurality of subsets of test routines are selected for a plurality of functional circuit components.

23. The method of claim 22 wherein each functional circuit component is tested by a corresponding subset of test routines independently of other functional circuit components.

24. The method of claim 22 wherein each subset of test routines is associated with instructions for turning on a corresponding functional circuit component and turning off one or more other functional circuit components.

25. An integrated circuit comprising:
a plurality of circuit modules corresponding to analog functional circuit components, the plurality of circuit modules comprising analog circuits;
a plurality of switch circuits, the plurality of switch circuits comprising:
a first plurality of switch circuits configured between inputs of corresponding circuit modules and outputs of preceding circuit modules;
a second plurality of switch circuits configured between outputs of the corresponding circuit modules and inputs of a subsequent circuit modules;
a third plurality of switch circuits configured between the inputs of the corresponding circuit modules and at least one analog test input; and
a fourth plurality of switch circuits configured between analog nodes of the corresponding circuit modules and at least one analog test output; and
one or more digital configuration circuits comprising:
a plurality of digital inputs configured to receive digital signals from an external source; and
a plurality of digital outputs coupled to the plurality of switch circuits,
wherein, during normal operation, the first plurality of switch circuits and second plurality of switch circuits are closed and the third plurality of switch circuits and fourth plurality of switch circuits are open, and
wherein, during testing, the first plurality of switch circuits and second plurality of switch circuits are open and the third plurality of switch circuits and fourth plurality of switch circuits are closed.

26. The integrated circuit of claim 25 wherein, during testing, the at least one analog test input is coupled to an external source and the at least one analog test output is couple to a measurement system.

27. The integrated circuit of claim 25 wherein one or more of the analog nodes are an internal nodes.

28. The integrated circuit of claim 25 wherein one or more of the analog nodes are outputs of the corresponding circuit modules.

29. The integrated circuit of claim 28, the plurality of switch circuits further comprising a fifth plurality of switch circuits, the fifth plurality of switch circuits configured between internal analog nodes of the corresponding circuit modules and the at least one analog test output.

30. The integrated circuit of claim 25 wherein the third plurality of switch circuits are selectively coupled to the at least one analog test input through a sixth plurality of switch circuits.

31. The integrated circuit of claim 30 wherein the sixth plurality of switch circuits form one or more analog multiplexers.

32. The integrated circuit of claim 30 wherein the sixth plurality of switch circuits form an analog tree structure between the at least one analog test input and the third plurality of switch circuits.

33. The integrated circuit of claim 25 wherein the fourth plurality of switch circuits are selectively coupled to the at least one analog test output through a sixth plurality of switch circuits.

34. The integrated circuit of claim 33 wherein the sixth plurality of switch circuits form one or more analog multiplexers.

35. The integrated circuit of claim 33 wherein the sixth plurality of switch circuits form an analog tree structure between the fourth plurality of switch circuits the at least one analog test output.

36. The integrated circuit of claim 25 wherein at least a portion of the switch circuits coupled to each circuit module are configured by one or more corresponding addressable digital configuration circuits.

37. The integrated circuit of claim 36 wherein the digital configuration circuits each comprise an address, and wherein switch circuits of the plurality of switch circuits coupled to a first circuit module are opened and closed in response to one or more corresponding digital configuration circuits receiving a first address and data.

38. The integrated circuit of claim 25 wherein the digital configuration circuits are coupled to a synchronous bus interface circuit to receive data from an external source.

39. A computer-implemented method comprising:
receiving, by at least one software system executing on at least one computer, circuit specification parameters corresponding to at least one analog functional circuit component the parameters comprising an activated test mode parameter;
selecting, based on the parameters and the activated test mode parameter, a plurality of subcircuit schematics, wherein the subcircuit schematics include circuitry for externally accessing the at least one functional circuit component when the at least one functional circuit component is embedded in a circuit design; and
generating a transistor level schematic for the circuit design, the transistor level schematic comprising circuitry for accessing one or more of an input node, an output node, or an internal node of the at least one analog functional circuit component.

40. The method of claim 39 wherein a different plurality of subcircuit schematics are selected when the test mode parameter is deactivated.

41. The method of claim 39 further comprising selecting a subset of test routines from a plurality of test routines for the at least one analog functional circuit component, wherein the plurality of test routines are configured to test the at least one analog functional circuit component across different values of the circuit specification parameters.

42. A computer-implemented method comprising:
generating a plurality of circuit designs from a plurality of functional circuit components, the functional circuit components comprising a plurality of parameters, wherein the circuit designs have at least some different circuit modules corresponding to different functional circuit components having different parameters;
selecting, for the functional circuit components in the circuit designs, a plurality of test routines, wherein the test routines are selected based, at least in part, on parameter values of the functional circuit component parameters;
gathering a plurality of test data for the plurality of circuit designs, the test data comprising results of a plurality of tests for each functional circuit component;
storing the plurality of test data in a database; and
updating behavioral models for the functional circuit components using the test data.

43. A computer-implemented method comprising:
specifying a plurality of functional circuit components for a circuit to be generated;
for each of one or more of the functional circuit components of the plurality of functional circuit components:
specifying parameters of the functional circuit component; and
selecting, based on the parameters, a predefined behavioral model corresponding to the functional circuit component having the specified parameters, wherein different parameters for a same functional circuit component result in selection of different corresponding behavioral models; and
executing a behavioral simulation of the circuit to be generated based on the selected predefined behavior models for the plurality of functional circuit components.

44. The method of claim 43 further comprising generating a mask for the circuit, wherein the mask is generated based on the behavioral simulation of the circuit and not a transistor level simulation of the circuit.

45. The method of claim 43 wherein a plurality of predefined behavioral models corresponding to particular functional circuit components having different user specified parameters are generated from characterization data from one or more automatically generated circuits comprising said functional circuit components.

46. The method of claim 43 further comprising:
receiving data specifying the physical behavior of at least one of the plurality of functional circuit components having particular specified parameters; and
modifying one or more behavioral models corresponding to particular functional circuit components having particular specified parameters based on at least a portion of the data.

47. The method of claim 43 wherein the parameters are generated in response to user inputs.

48. The method of claim 43 wherein the functional circuit components are analog functional circuit components for generating analog circuits.

49. The method of claim 43 wherein the functional circuit components have a plurality of corresponding parameters, wherein different values for each of the plurality of corresponding parameters produce functional circuit components with different characteristics.

50. The method of claim 43 wherein the functional circuit components are one or more of: a comparator circuit, an oscillator circuit, a delay circuit, a current generator circuit, a voltage reference circuit, an amplifier circuit, a voltage buffer circuit, a bandgap circuit, a current mirror circuit, a transconductance circuit, and a voltage-to-current converter circuit.

51. A method comprising:
specifying a plurality of functional circuit components for a circuit to be generated;
specifying parameters of the plurality of functional circuit components;
generating a mask for the circuit to be generated comprising the plurality of functional circuit components having the specified parameters;
fabricating the circuit based on the mask;
storing data specifying an actual behavior of the plurality of functional circuit components having the specified parameters in the fabricated circuit; and
for each functional circuit component, generating a customized behavioral model corresponding to the functional circuit component having the specified parameters based on at least a portion of the data.

52. The method of claim 51 wherein the plurality of functional circuit components each having corresponding predefined behavioral models, and wherein the predefined behavioral models are modified based on said at least a portion of the data to generate the customized behavioral models corresponding to the specified parameters.

53. The method of claim 52 wherein the predefined behavioral models are modified based on at least a portion of the data to generate customized behavioral models corresponding to unique combinations of parameters.

54. The method of claim 51 further comprising:
specifying a second plurality of functional circuit components for a second circuit to be generated;
for each of one or more of the second plurality of functional circuit components:
specifying parameters of a particular functional circuit component; and
selecting, based on the parameters, a particular predefined parameterized behavioral model corresponding to the functional circuit component having the specified parameters, wherein different parameters for a same functional circuit component result in selection of different corresponding predefined parameterized behavioral models; and
executing a behavioral simulation of the second circuit to be generated based on one or more predefined parameterized behavior models for the plurality of functional circuit components.

55. The method of claim 54 further comprising generating a mask for the second circuit, wherein the mask is generated based on a behavioral simulation of the circuit and not a transistor level simulation of the circuit.

56. The method of claim 51 wherein the parameters are user specified.

57. The method of claim 51 wherein the functional circuit components are analog functional circuit components for generating analog circuits.

58. The method of claim 51 wherein the functional circuit components have a plurality of corresponding parameters, wherein different values for each of the plurality of corresponding parameters produce functional circuit components with different characteristics.

59. The method of claim 51 wherein the functional circuit components are one or more of: a comparator circuit, an oscillator circuit, a delay circuit, a current generator circuit, a voltage reference circuit, an amplifier circuit, a voltage buffer circuit, a bandgap circuit, a current mirror circuit, a transconductance circuit, and a voltage-to-current converter circuit.

60. The method of claim 51 wherein the data is test data for the circuit representing an actual behavior of the circuit.
